(19) United States
(12) Patent Application Publication
(10) Pub. No.: US 2024/0390499 A1
(43) Pub. Date: Nov. 28, 2024

(54) METHOD

(71) Applicants: OSLO UNIVERSITETSSYKEHUS HF, Oslo (NO); NATIONAL AND KAPODISTRIAN UNIVERSITY OF ATHENS, Athens (GR)

(72) Inventors: Georgios C. VOUGIOUKALAKIS, Attiki (GR); Georgios ROTAS, Koropi Attikis (GR); Theodossis THEODOSSIOU, Oslo (NO); Kristian BERG, Heggedal (NO); Miguel Angel Miranda ALONSO, Valencia (ES); Mantas GRIGALAVICIUS, Gjetum (NO); Somayeh EZZATPANAH, Snarøya (NO); Tine Therese Henriksen RAABE, Oslo (NO)

(21) Appl. No.: 18/693,680

(22) PCT Filed: Sep. 20, 2022
(86) PCT No.: PCT/GB2022/052366

§ 371 Date: Mar. 20, 2024

(30) Foreign Application Priority Data
Sep. 20, 2021 (GR) 20210100622
Feb. 1, 2022 (EP) 22154473.7

(51) Int. Cl. A61K 41/00

(52) U.S. Cl. CPC A61K 41/0057; A61K 41/0061

(57) ABSTRACT

The invention provides endoplasmic reticulum-targeted chemiluminescent agents and their use in methods of photodynamic therapy (PDT). In particular, the invention provides compounds of general formula (I), and their pharmaceutically acceptable salts: in which A represents a chemiluminescent moiety; each L, which may be the same or different, is either a direct bond or a linker; each B, which may be the same or different, represents an endoplasmic reticulum-targeting moiety; n is an integer from 1 to 3, preferably 1; and x is an integer from 1 to 3, preferably 1. Such compounds find particular use in the treatment of deeply-sited tumours, e.g. glioblastoma multiforme (GBM), when used in combination with a photosensitizer or photosensitizer precursor.

### TECHNICAL FIELD

The present invention relates to improvements in and relating to methods of photodynamic therapy (PDT) and, in particular, to such methods for the targeted treatment of diseases and conditions characterised by hyperproliferative and/or abnormal cells, without the need for an external light source. More specifically, the invention relates to such methods for the treatment of tumours, especially those which are inaccessible when using existing PDT methods.

The invention further relates to novel endoplasmic reticulum-targeted chemiluminescent agents, to methods for their preparation and to their use as an intracellular light source in methods of PDT which employ a photosensitizer or photosensitizer precursor.

### BACKGROUND OF THE INVENTION

Conventional treatment of internal tumours typically involves invasive surgery, radiotherapy, non-curative chemotherapy, or a combination of these. Intracranial tumours such as glioblastoma multiforme (GBM) are one example of deeply-sited tumours which are very difficult to treat because of their location and highly aggressive characteristics. Approximately 28,000 new cases of malignant glioma such as GBM are diagnosed every year in the EU and the US and in 240,000 patients globally every year. The current standard therapy consists of highly invasive (open brain) surgery which removes about 99% of the tumour but leaves behind about a billion cells, leading to recurrence. Radiotherapy may be used as an adjuvant to surgery (at 60-65 Gy) and together with surgery may reduce the cancer cells left behind to several million, however radiotherapy does not have a major effect on cancers such as GBM which tend to spread in several locations also harbouring radio-resistant cancer cells. Furthermore, radiotherapy is not specific in destroying cancerous vs. normal tissues. Chemotherapy with temozolomide in addition to radiotherapy may also be used. However, these therapies offer limited overall patient survival and do not produce a curative outcome; these are mainly cytostatic as cells will eventually (within approx. 1 year of treatment) develop resistance and render the treatment no longer effective. The combination of surgery with radiotherapy increases the median of survival from 4.5 months (untreated) to 12.1 months. Additional chemotherapy with temozolomide extends survival to 14.6 months. The relative survival rate for adults diagnosed with GBM is less than 30% within one year of diagnosis and only 3% of patients live longer than five years after initial diagnosis.

Deep lying, hard to reach tumours such as GBM thus remain very difficult to treat and existing therapies offer only a minimal increase in survival rates. Therefore, the development of more targeted and less invasive therapeutic approaches with improved efficacy is urgently required.

Other methods known for use in the treatment of tumours include PDT. PDT involves the administration of a photosensitizer, either locally or systemically, followed by exposure of the affected area to photoactivating light which interacts with the ambient oxygen to produce cytotoxic intermediates. This results in the destruction of cells and the shutdown of the tumour vasculature.

PDT provides cancer treatment through the synergy of three essential, yet individually non-chemotoxic, components: (i) the photosensitizer (PS), a light activated drug; (ii) light of the appropriate wavelength to activate the PS; and (iii) the presence of oxygen, which is the terminal generator of toxic species. The anti-tumour effects of PDT can mainly be categorized into three interrelated effects: (i) direct cytotoxic action which is mainly effected through either a type I or type II mechanism—the former generates reactive oxygen species (ROS) and ultimately hydroxyl radicals while a type II mechanism, prominent in the majority of PSs, generates deleterious singlet oxygen [O₂ (¹Δ_{g}) or ¹O₂]; (ii) damage to tumour vasculature; and (iii) induction of an inflammatory reaction that can lead to the development of systemic immunity, as a consequence of PDT-induced oxidative stress.

Photosensitizing agents which are currently approved for use in methods of photodynamic therapy and diagnosis include protoporphyrin IX (PpIX) which is produced from its biosynthetic non-photosensitive precursor 5-aminolevulinic acid (5-ALA). Following the external administration of 5-ALA, the biosynthetic cycle of heme facilitates its conversion to the active photosensitizer PpIX in cell mitochondria. Cancer cells treated with 5-ALA accumulate larger amounts of PpIX mainly due to their higher amount of PpIX-synthesis-promoting enzymes and/or substantially lower amount of ferrochelatase (this enzyme catalyses the chelation of iron by PpIX in the mitochondrial matrix to produce heme which is then transported out of the mitochondria). On subsequent exposure to light, PpIX is excited from its ground singlet state to its excited singlet state. It then undergoes intersystem crossing to a longer-lived excited triplet state. Upon interaction of the PpIX in the triplet state with an oxygen molecule (in the ground triplet state), an energy transfer takes place from the PpIX to oxygen. This results in a mutual spin flip of the two molecules which allows the PpIX to relax back to its ground singlet state, whilst creating an excited singlet state oxygen molecule which is cytotoxic.

Photosensitizing agents are also known for use in methods of photodynamic diagnosis of cancerous cells and tissues and can also be used to guide surgical resection of tumour masses. For example, PDT is used as an aid to surgery in the treatment of bladder cancer. 5-ALA induced PpIX fluorescence is currently also used intraoperatively for fluorescence guided resection in the treatment of GBM (see Stummer et al., Lancet Oncol., 2006, 7(5): 392-401). However, due to limitations of conventional PDT procedures (e.g. light accessibility and light penetration into tissue) it cannot, at present, provide any significant benefit in treating this aggressive condition without the need for surgical intervention.

The main limitations of existing methods of PDT as an anti-cancer treatment are poor determination of the treated area by the clinician, poor definition of treated tumour volume, and the limited depth of penetration of the photoactivating light in tissue (˜1.5 cm). This leads to ineffective treatment and viable cancer cells being left behind. 5-ALA based PDT, for example, is highly specific and efficient for the treatment of actinic keratosis and basal cell carcinoma with a high cure rate, but only for lesions thinner than 2 mm. For thicker lesions or non-superficial cancers, 5-ALA PDT cannot guarantee the patient a cure, and in cases of large tumours it is merely palliative. This is due to the limited tissue penetration of light at the wavelength of PpIX activation (635 nm).

Whilst in some cases PDT may be used to treat deeper seated tumours in solid organs or hollow organs like the oesophagus, this generally involves the use of a device, such as a catheter- or endoscope-directed fibre optic, for light activation of the photosensitizer. Not only is this a complicated procedure, but it precludes access to certain areas of the body and introduces a level of invasiveness to the treatment. It also cannot eradicate the entirety of the cancer cells, and cannot be applied to multi-foci diseases (e.g. gliomas) or multi-foci metastases. Thus, although appropriate for treating superficial tumours, the use of existing PDT methods in treating deeply seated tumour cells and anatomically less accessible lesions is severely limited.

Since the main limitation of PDT is the access of light to the cancerous lesions, especially when these are in deep lying organs like the brain, liver or pancreas, several efforts have been made to utilise bio- or chemi-luminescence as intracellular sources which would provide the light needed for a photodynamic cell suicide following administration of the photosensitizer. One such treatment, initially developed in 2013 (Theodossiou et al., Cancer Research, 2013, 63:1818-21), is BLADe (BioLuminescence Activated Destruction). BLADe relies on the intracellular transfection with the firefly luciferase enzyme and the subsequent administration of a photosensitizer and luciferin, the natural substrate of luciferase. The main shortcomings of this method are the need for genetic modification of the cells to produce luciferase and the requirement for co-localisation of the above three factors and ATP. Also, this co-localisation has to be in the very close vicinity of vulnerable, intracellular singlet oxygen targets.

Several attempts to exploit luminescence in order to achieve the desirable PDT effect have since been made (see, for example, Hsu et al., Biomaterials, 2013, 34(4): 1204-12; and Baacirova et al., Luminescence, 2011, 26(6): 410-5). Laptev et al. (Br. J. Cancer, 2006, 95(2): 189-96) have previously proposed the use of luminol (5-amino-2,3-dihydro-1,4-phthalazinedione) together with transferrin-haematoporphyrin conjugates to kill cells by intracellular luminescence. Although they provided sufficient proof-of-concept (95% cytotoxicity), the following shortcomings are associated with these methods making them non-viable in the clinic: (i) non-specific intracellular targeting; (ii) in the work by Laptev et al., the need for transferrin as the iron source; and (iii) the lack of design for proximity to intracellular ROS.

More recently, in WO 2019/243757, the inventors proposed the use of mitochondrial-targeted chemiluminescent agents in methods of PDT to treat tumors such as GBM. This earlier work involves chemical modification of luminol to attach groups which target it to cell mitochondria and which efficiently transport it across the mitochondrial membrane. Mitochondrial respiration provides the reactive oxygen species (ROS) and transition metal catalysts necessary for luminol luminescence. Moreover, PpIX is biosynthesised in mitochondria and is thus in close proximity to the luminol for efficient activation and deleterious singlet oxygen production which kills the host cells. In this way, the mitochondrial ROS which do not pose an immediate threat to cell survival, are “upgraded” to a highly cytotoxic ROS which inflicts fatal cell damage from within the cell. This action is specific to the cancerous lesion due to the production of high levels of PpIX at the target site. The chemically modified luminol ('mitotropic luminol') is thus employed as a self-sustained, intracellular source of light and the target cell mitochondria are used as the power supply for “switching on the light”. This consequently activates the cytotoxic activity of the photosensitizer (e.g. PpIX) within the tumour cells.

In a development of their earlier work, the inventors now propose an alternative, non-invasive method of PDT involving the use of chemically-modified chemiluminescent agents, such as luminol, that are capable of accumulating in the endoplasmic reticulum (ER) of the target tumour cells.

Although efficient in tumour cell killing, the mitochondria-targeting compounds in WO 2019/243757 may have a limited therapeutic window before they reach toxic concentrations. This is due to the dissipation of the mitochondrial membrane potential as a result of accumulation of the cationic charges of the compounds in the mitochondrial matrix. In the case of ER-targeting, however, the tolerance is much higher as there is no such effect. At the same time, the endoplasmic reticulum is amongst the vulnerable intracellular PDT targets and, due to its numerous contact sites with mitochondria, it offers proximity to mitochondrial-produced ROS (Vance, Biochim. Biophys. Acta, 2014, 1841:595-609). Furthermore, the agents now proposed for use in PDT may also utilize ER-produced ROS [doi: 10.1089/ars.2014.5851], especially that from the unfolded protein response (UPR) which is vital for the progression and survival of cancers [doi: 10.1111/boc.201800050]). It is expected that these agents may therefore achieve a more efficient killing of tumour cells and thus provide an improved treatment over that described in WO 2019/243757.

Whilst particularly suitable for the treatment of internal tumour masses which cannot be accessed using conventional PDT techniques, the method of PDT herein described finds use in the treatment of all tumour types and any conditions which involve hyperproliferation of cells.

### SUMMARY OF THE INVENTION

The invention relates to a method of PDT which involves the combined use of a photosensitizer, or a precursor of a photosensitizer (e.g. 5-ALA), and a chemiluminescent agent which is capable of targeting the endoplasmic reticulum (“ER”). Affinity for the endoplasmic reticulum is achieved by the use of a “modified” chemiluminescent agent, specifically a chemiluminescent agent ‘conjugate’ in which at least one chemiluminescent moiety is bound to at least one endoplasmic reticulum-targeting moiety (also referred to herein as an “ER-targeting moiety”).

Examples of chemiluminescent agent ‘conjugates’ are chemically modified luminols and acridinium esters. Their chemical modification involves the attachment of one or more chemical groups which target them to the endoplasmic reticulum.

Broadly speaking, the invention thus involves the modification of known chemiluminescent agents such that these are ER-targeted to carry out PDT specifically on hyperproliferative and/or abnormal cells, e.g. cancer cells. As a result of this modification, the luminescence required to activate photosensitizers that accumulate at the target site, such as cercosporin, hypericin and PpIX (following its translocation from the mitochondria), is ‘automatic’ and even more intense in cancer cells which, in many cases, exhibit higher mitochondrial and ER ROS formation.

For example, 5-ALA derived PpIX formation is highly specific to the cancerous GBM lesion. This high specificity leads to the possibility of a GBM treatment where no invasive approach is required, but just the systemic administration of an ER-targeted chemiluminescent agent, such as ER-targeted luminol, and 5-ALA is sufficient to eradicate all GMB lesions in the brain. This is especially important since GBM is highly migratory within the brain following its initial occurrence and resurfaces at different brain locations. When used to treat GBM, the invention also takes advantage of the GBM-induced destruction of the blood-brain barrier so that both the modified chemiluminescent agent (e.g. luminol or an acridinium ester) and the photosensitizer or precursor thereof (e.g. cercosporin or 5-ALA) reach the GMB lesions in the brain efficiently.

In the invention, PDT is effectively applied to each individual tumour cell (i.e. the PDT effect is at the single cell level, rather than the collective lesion) without the requirement for an external light source, such as a lamp or a laser which is conventionally used in PDT. In this therapeutic approach to PDT, the depth of light penetration into tissue is no longer a limitation. It establishes the basis for an alternative treatment of cancer which, despite being photochemical, involves the administration of two individually non-chemotherapeutic drugs. As such, it can be repeated multiple times without the risk of adverse side-effects. This minimises the risk of metastasis and maximises the curative potential of the treatment.

The invention further addresses the need for an effective treatment of internal cancers, such as GBM, which at present are practically incurable due to their location and highly aggressive nature, without the need for invasive surgery, ionizing radiation or non-curative chemotherapy. Such treatment may be either as a primary treatment and/or as a photochemotherapeutic which is able to effectively control the progression of the disease for life through repeated, non-invasive, treatments. This approach to the treatment of inaccessible cancers extends to the treatment of other diseases and conditions which are characterised by hyperproliferative and/or abnormal cells, and, in particular, to the treatment of other cancers including those which are shallow or superficial.

### DETAILED DESCRIPTION OF THE INVENTION

### Definitions

As used herein, the term “chemiluminescent agent” is intended to encompass any of a variety of agents which are capable of emitting light as a result of a chemical reaction which takes place within the endoplasmic reticulum (ER) of the cell, i.e. any agent which can be ‘activated’ once localised in the ER. More specifically, the chemiluminescent agent will be an agent which emits light following reaction with a substance which is either present in the endoplasmic reticulum of the cell or generated in close proximity thereto (e.g. in the neighbouring cell mitochondria or other intracellular sources), such as a reactive oxygen or nitrogen species. Such oxygen species include, for example, any reactive oxygen species (ROS) such as oxygen radicals, oxygen superoxide anion, hydroxyl radicals, etc., and hydrogen peroxide. Nitrogen species include, for example, nitric oxide, peroxynitrite, nitrogen oxides, etc. As will be understood, any chemiluminescent agent for use in the invention will be physiologically tolerable.

As used herein, the term “chemiluminescent moiety” encompasses any chemiluminescent agent or any moiety derived from a chemiluminescent agent, i.e. a derivative thereof. Any derivative should retain the light-emitting properties of the parent molecule as noted above. This should similarly meet the requirement of physiological tolerability in vivo. Examples of derivatives include chemiluminescent agents carrying one or more additional functional or non-functional groups (e.g. substituents). The term “derivative” also extends to a fragment or residue of a chemiluminescent agent.

The terms “endoplasmic reticulum-targeting moiety” and “ER-targeting moiety” are used interchangeably herein and are intended to encompass any physiologically acceptable agent or moiety which is capable of targeting and hence accumulating in the endoplasmic reticulum. It also encompasses derivatives of such agents which retain the endoplasmic reticulum-targeting properties of the parent molecule. The term “derivative” extends to a fragment or residue of an endoplasmic reticulum-targeting agent.

As used herein, the term “photosensitizer precursor” is intended to encompass any compound which is converted metabolically to a photosensitizer and is thus essentially equivalent thereto.

The term “pharmaceutically acceptable salt” as used herein refers to any pharmaceutically acceptable organic or inorganic salt of any of the compounds herein described. A pharmaceutically acceptable salt may include one or more additional molecules such as counter-ions. The counter-ions may be any organic or inorganic group which stabilizes the charge on the parent compound. If the compound is a base, a suitable pharmaceutically acceptable salt may be prepared by reaction of the free base with an organic or inorganic acid. If the compound is an acid, a suitable pharmaceutically acceptable salt may be prepared by reaction of the free acid with an organic or inorganic base.

The term “pharmaceutically acceptable” means that the compound or composition is chemically and/or toxicologically compatible with other components of the formulation or with the patient (e.g. human) to be treated.

By “a pharmaceutical composition” is meant a composition in any form suitable to be used for a medical purpose.

As used herein, the term “treatment” includes any therapeutic application that can benefit a human or non-human animal (e.g. a non-human mammal). Both human and veterinary treatments are within the scope of the present invention, although primarily the invention is aimed at the treatment of humans. The term “treatment” or “therapy” encompasses curative as well as prophylactic treatment or therapy.

The term “alkyl” as used herein refers to a monovalent saturated, linear or branched, carbon chain. Examples of alkyl groups include, but are not limited to, methyl, ethyl, n-propyl, iso-propyl, n-butyl, iso-butyl, sec-butyl, tert-butyl, n-pentyl, iso-pentyl, neo-pentyl, n-hexyl, etc. An alkyl group preferably contains from 1-6 carbon atoms, e.g. 1-4 carbon atoms.

The term “alkoxy” as used herein refers to an —O-alkyl group, wherein alkyl is as defined herein. Examples of alkoxy groups include, but are not limited to, methoxy, ethoxy, propyloxy, etc.

The term “aryl” as used herein refers to aromatic ring systems. Such ring systems may be monocyclic or bicyclic and contain at least one unsaturated aromatic ring. Where these contain bicyclic rings, these may be fused. Preferably such systems contain from 6-20 carbon atoms, e.g. either 6 or 10 carbon atoms. Examples of such groups include phenyl, 1-naphthyl and 2-naphthyl. A preferred aryl group is phenyl. Unless stated otherwise, any aryl group may be substituted by one or more substituents selected from hydroxy, C₁₋₆ alkyl, C₁₋₆ alkoxy, amino, cyano, or nitro groups, or halogen atoms (e.g. F, Cl or Br). Where more than one substituent group is present, these may be the same or different.

The term “halogen atom” refers to F, Cl, Br or I.

The term “haloalkyl” as used herein refers to an alkyl group as defined herein in which at least one of the hydrogen atoms of the alkyl group is replaced by a halogen atom, preferably F, Cl or Br. Examples of such groups include —CH₂F, —CHF₂, —CF₃, —CCl₃, —CHCl₂, —CH₂CF₃, etc.

The term “heterocyclic ring” as used herein refers to a saturated or partially unsaturated, 4- to 6-membered (preferably 5- or 6-membered) carbocyclic system in which at least one ring atom is a heteroatom selected from nitrogen, oxygen and sulfur, the remaining ring atoms being carbon. The heterocyclic ring structure may be linked to the remainder of the molecule through a carbon atom or through a nitrogen atom. Unless otherwise stated, any heterocyclic ring mentioned herein may optionally be substituted by one or more groups, which may be identical or different, for example hydroxy, C₁₋₆ alkyl, C₁₋₆ alkoxy, amino, cyano, or nitro groups, or halogen atoms (e.g. F, Cl or Br).

Unless otherwise stated, all substituents are independent of one another.

In the case where a subscript is the integer 0 (i.e. zero), it is intended that the group to which the subscript refers is absent.

In one aspect the invention provides an endoplasmic reticulum-targeted chemiluminescent agent for use in a method of photodynamic therapy.

In one embodiment the endoplasmic reticulum-targeted chemiluminescent agent for use in the invention is a chemiluminescent agent ‘conjugate’ which comprises at least one chemiluminescent moiety attached to or otherwise associated with at least one endoplasmic reticulum-targeting moiety (“ER-targeting moiety”) that selectively targets the endoplasmic reticulum. Where this conjugate comprises more than one chemiluminescent moiety, these may be the same or different. Generally, however, these will be identical. Where the chemiluminescent moiety is attached to more than one ER-targeting moiety, the ER-targeting moieties may be the same or different, but preferably will be the same. In one embodiment, the conjugate comprises a single chemiluminescent moiety attached to or otherwise associated with a single ER-targeting moiety.

The chemiluminescent moiety (or moieties) may be attached to the ER-targeting moiety (or moieties) through covalent or non-covalent means. It may, for example, be bound via electrostatic interaction, van der Waals forces and/or hydrogen bonding. Typically, the chemiluminescent moiety (or moieties) and ER-targeting moiety (or moieties) will be covalently bound to one another, for example via one or more covalent bonds. In some cases, the chemiluminescent moiety (or moieties) may be covalently bound to the (or each) ER-targeting moiety via a linking group (or “spacer”).

The chemiluminescent agent ‘conjugate’ for use in the invention may be a compound having the following general formula (I), or a pharmaceutically acceptable salt thereof:

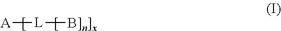

in which A represents a chemiluminescent moiety;
each L, which may be the same or different, is either a direct bond or a linker;
each B, which may be the same or different, represents an endoplasmic reticulum-targeting moiety;
n is an integer from 1 to 3, preferably 1; and
x is an integer from 1 to 3, preferably 1.

In one embodiment of formula (I), both n and x are 1. The chemiluminescent agent ‘conjugate’ for use in the invention may therefore be a compound of formula (II), or a pharmaceutically acceptable salt thereof:

A-L-B (II)

in which A, L and B are as herein defined.

Chemiluminescent agents suitable for use in the invention are known in the art and include, for example, luminol, isoluminol, lucigenin, acridinium esters, oxalate esters, and known analogues and derivatives thereof. Any known chemiluminescent agent or a derivative thereof may also be used. Chemiluminescent moieties for use in the invention may be ‘derived’ from any of these agents. Suitable derivatives may include one or more additional functional or non-functional groups (e.g. substituents), or these may comprise a fragment or residue of such agents which retain their chemiluminescent activity.

Preferred for use in the invention are chemiluminescent moieties which are derived from luminol, isoluminol, and acridinium esters. Such compounds may be substituted by one or more additional substituents, for example C₁₋₆ alkyl, C₁₋₆ alkoxy, amino, cyano, nitro and aryl (e.g. phenyl) groups, or halogen atoms. For example, the phenyl ring in luminol or isoluminol may be substituted by one or more (e.g. one or two) groups selected from halogen atoms, C₁₋₆ alkyl and phenyl groups.

As will be understood, in order to bind to the ER-targeting moiety (or moieties) via a covalent bond or linker, the chemiluminescent moiety will typically be a “derivative” of the parent chemiluminescent agent. For example, this may be devoid of one or more terminal atoms or groups following formation of a covalent bond either to the linker or directly to the ER-targeting agent and thus be considered a “residue” of the original molecule. For example, in the case of luminol the primary amine group may form the point of attachment either to the linker or the ER-targeting moiety and so it is ‘derivatised’ by the loss of a single hydrogen atom, i.e. —NH₂ to —NH—. Other forms of derivatisation may be envisaged, including the introduction of functional groups which may react to form a covalent bond with the ER-targeting moiety (or moieties). The particular form of ‘derivatisation’ required for any given chemiluminescent agent will be dependent on its structure and can readily be determined by any skilled chemist.

In formula (I) or (II), the chemiluminescent moiety may be selected from the following structures (in which * denotes its point (or points) of attachment to a linker, L, or directly to an ER-targeting moiety):

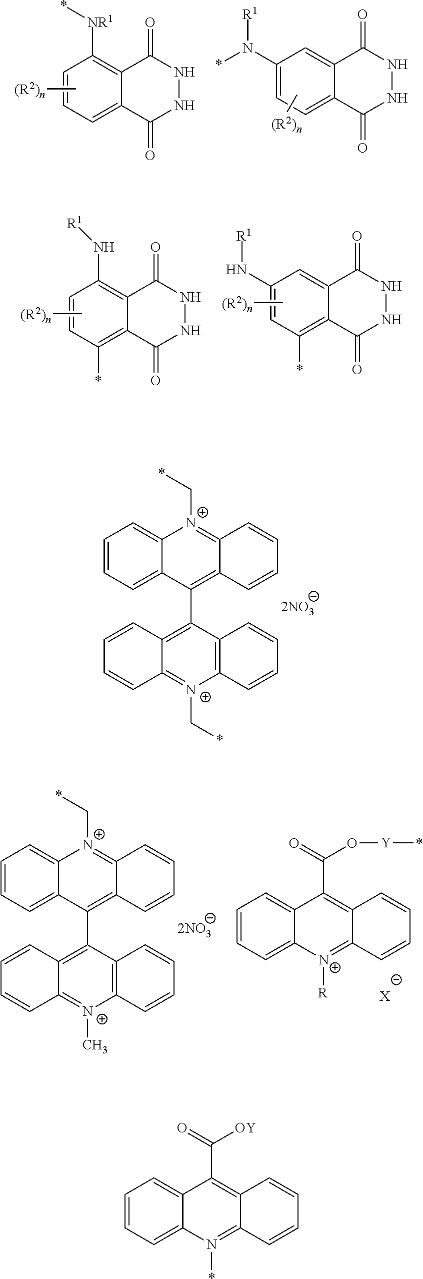

wherein:
R¹ is hydrogen or an alkyl group such as C₁₋₆ alkyl, preferably C₁₋₃ alkyl (e.g. methyl); each R² is independently selected from:
   C₁₋₆ alkyl,
   C₁₋₆ alkoxy,
   —NR⁵R⁶ (in which R⁵ and R⁶ are independently selected from H and C₁₋₆ alkyl, preferably from H and C₁₋₃ alkyl, e.g. —CH₃), and
   optionally substituted aryl (e.g. optionally substituted phenyl);
n is an integer from 0 to 3, preferably 0, 1 or 2, e.g. 0 or 1;
m is an integer from 0 to 2, e.g. 0 or 1;
R is hydrogen or C₁₋₆ alkyl, preferably C₁₋₃ alkyl (e.g. methyl);
X is a monovalent anion, e.g. a Cl, Br, I, OTos, ClO₄, NO₃, PF₆, or BF₄ anion; and
Y is an optionally substituted aryl (or arylene) group, e.g. optionally substituted phenyl (or phenylene).

In one embodiment, R¹ is hydrogen or methyl. Preferably, R¹ is hydrogen.

In one embodiment, each R² is independently selected from C₁₋₆ alkyl and unsubstituted phenyl. For example, each R² may be independently selected from methyl, ethyl, n-propyl, iso-propyl, n-butyl and phenyl.

Where Y is a substituted aryl or arylene group, examples of suitable substituents include one or more halogen atoms (e.g. F, Cl, Br, I), C₁₋₆ alkyl (e.g. tert.butyl, propyl, ethyl or methyl), —COOC₁₋₆ alkyl (e.g. —COOCH₃), nitro or cyano groups.

Different substitution patterns on the aromatic ring of luminol or isoluminol may be selected by those skilled in the art and, in one embodiment, these may be selected to enhance chemiluminescence. In one embodiment, the chemiluminescent moiety for use in the invention is luminol or a substituted derivative thereof, for example it may be a moiety having the following structure (in which * denotes the point of attachment to a linker, L, or directly to an ER-targeting moiety):

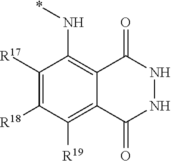

wherein:
R¹⁷ is hydrogen, C₁₋₆ alkyl (preferably C₁₋₄ alkyl, more preferably C₁₋₃ alkyl, e.g. methyl), or optionally substituted aryl (e.g. optionally substituted phenyl);
R¹⁸ is hydrogen or C₁₋₆ alkyl (preferably C₁₋₄ alkyl, more preferably C₁₋₃ alkyl, e.g. methyl); and
R¹⁹ is hydrogen or C₁₋₆ alkyl (preferably C₁₋₄ alkyl, more preferably C₁₋₃ alkyl, e.g. methyl).

In one embodiment, R¹⁷ and R¹⁸ are both hydrogen and R¹⁹ is other than hydrogen, for example R¹⁹ is C₁₋₃ alkyl, e.g. methyl.

In another embodiment, R¹⁸ is hydrogen and each of R¹⁷ and R¹⁹ are other than hydrogen, for example R¹⁷ is optionally substituted phenyl, and R¹⁹ is C₁₋₆ alkyl (e.g. methyl).

Non-limiting examples of chemiluminescent moieties for use in the invention include the following (in which * denotes the point of attachment to a linker, L, or directly to an ER-targeting moiety):

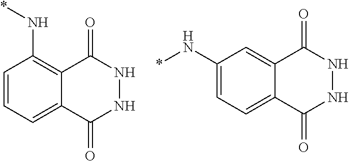

Any agent known to be capable of selectively targeting the endoplasmic reticulum may be used to provide the “ER-targeting moiety” in the conjugates herein described. Such agents will typically be hydrophobic in nature to make them compatible for partitioning into the ER membrane environment. Particularly suitable for use in the invention are sulphonamide agents due to their ability to localise in the endoplasmic reticulum. Such agents carry one or more sulphonamide groups. Typically, these will comprise one sulphonamide group.

As will be understood, in order to bind to the chemiluminescent moiety (or chemiluminescent moieties) via a covalent bond or linker, the ER-targeting moiety may, in some cases, be a ‘derivative’ of the parent agent. For example, this may be devoid of one or more terminal atoms or groups following formation of a covalent bond either to the linker or directly to the chemiluminescent agent (or agents) and thus be considered a “residue” of the original molecule. The particular form of ‘derivatisation’ required for any given ER-targeting moiety will be dependent on its structure and can readily be determined by any skilled chemist.

Sulphonamide ER-targeting moieties for use in the invention include the following (in which * denotes the point of attachment to a linker, L, or directly to a chemiluminescent moiety):

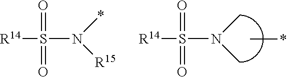

wherein
R¹⁴ is C₁₋₆ alkyl or an optionally substituted aryl group;
R¹⁵ is hydrogen or C₁₋₆ alkyl; and

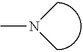

denotes an optionally substituted, nitrogen-containing heterocyclic ring.

In one embodiment, R¹⁴ is C₁₋₆ alkyl, preferably C₁₋₃ alkyl, e.g. —CH₃.

In one embodiment, R¹⁴ is an optionally substituted aryl group. For example, R¹⁴ may be optionally substituted phenyl or naphthyl.

Where R¹⁴ is substituted, it may be substituted by one or more substituent groups selected from the group consisting of C₁₋₃ alkyl (e.g. methyl), C₁₋₃ alkoxy (e.g. methoxy), C₁₋₃ haloalkyl (e.g. —CF₃), —OC₁₋₃ haloalkyl (e.g. —OCF₃), cyano, nitro, —NR′₂ (where each R′ is independently H or C₁₋₃ alkyl), halogen (e.g. F, Cl or Br), —COR″ (in which R″ is H or C₁₋₃ alkyl), and —COOR″ (in which R″ is H or C₁₋₃ alkyl). Preferred substituent groups are C₁₋₃ alkyl (e.g. methyl). Typically one or two substituent groups may be present, for example a single substituent group. Where a single substituent group is present on a phenyl ring, it may be in the ortho, meta or para position. In one embodiment, it will be present at the para position. For example, a methyl group may be present at the para position of the phenyl ring.

In one embodiment, R¹⁴ is an unsubstituted aryl group, for example unsubstituted phenyl or naphthyl.

In one embodiment, R¹⁵ is hydrogen or —CH₃, e.g. hydrogen.

In one embodiment,

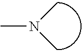

denotes a piperazinyl ring. This may be linked to a linker, L, or directly to a chemiluminescent moiety via a ring carbon or via the additional ring nitrogen atom, but is preferably linked via the additional ring nitrogen atom. In a preferred embodiment, the ER-targeting moiety may be represented as follows:

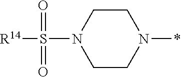

wherein
R¹⁴ is as herein defined; and
* denotes the point of attachment to a linker, L, or directly to a chemiluminescent moiety.

In one embodiment, the ER-targeting agent is a group having one of the following structures:

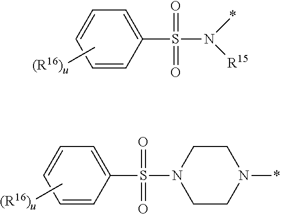

wherein
R¹⁵ is as herein defined;
each R¹⁶ independently represents C₁₋₃ alkyl (e.g. methyl), C₁₋₃ alkoxy (e.g. methoxy) or a halogen atom (e.g. Cl or Br);
u is an integer from 0 to 5, preferably 1 to 3, e.g. 1; and
* denotes the point of attachment to a linker, L, or directly to a chemiluminescent moiety.

Non-limiting examples of ER-targeting moieties for use in the invention include the following (in which * denotes the point of attachment to a linker, L, or directly to a chemiluminescent moiety):

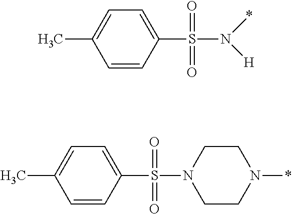 [SMILES: *NS(=O)(=O)c1ccc(C)cc1 ; *N1CCN(S(=O)(=O)c2ccc(C)cc2)CC1]

In general formulae (I) and (II) above, the precise nature of the linker, L, is generally not considered to be critical to performance of the invention provided that this serves its intended function of linking the chemiluminescent moiety to the ER-targeting moiety (or moieties) and thus enables the targeted delivery of the chemiluminescent moiety to the endoplasmic reticulum. The linker may be rigid or flexible and may be cleavable in vivo at the desired target site (e.g. it may be photocleavable). Generally, it will comprise organic groups.

The linking group, L, may be hydrophilic or hydrophobic in nature. It may either be branched (including dendritic) or straight-chained, but preferably it will be straight-chained. Where the linking group is branched this may, for example, carry more than one ER-targeting moiety. The linking group may be aliphatic and/or aromatic and may comprise one or more cycloalkyl, heterocyclic, aryl, or heteroaryl rings. The linking group may thus be aliphatic, (poly) cyclic and/or (poly) aromatic in nature.

The chain length of the linker may vary, although in general this may comprise a backbone containing from 1 to 20 atoms (e.g. 1 to 20 carbon atoms), preferably from 2 to 15, e.g. from 2 to 12 atoms. In some cases, the length of the linker may be varied to adjust the exact positioning of the chemiluminescent part of the conjugate relative to the ER-targeting moiety.

Linker L may, for example, comprise an alkylene chain (preferably a C₁₋₁₅ alkylene, e.g. a C₂₋₁₁ alkylene) optionally substituted by one or more groups selected from C₁₋₃ alkyl, —O(C₁₋₃)alkyl, —OH, cycloalkyl and aryl groups; and in which one or more —CH₂— groups of the alkylene chain may be replaced by a group independently selected from —O—, —CO—, —NR— (where R is H or C₁₋₆ alkyl, preferably C₁₋₃ alkyl, e.g. methyl), cycloalkyl, heterocyclic, aryl and heteroaryl groups. In one embodiment, all —CH₂— groups of the alkylene chain may be replaced by such groups. For example, the linker L may be a group —CO—.

Suitable linker groups may readily be determined by those skilled in the art. Examples of suitable linkers include optionally substituted alkylene groups, preferably unsubstituted, straight-chained alkylene groups, e.g. —C₃H₆—, —C₄H₈—, —C₆H₁₂—, —C₈H₁₆—, —C₁₀H₂₀—, and —C₁₁H₂₂—. In one embodiment, short chain alkylene groups are preferred such as —C₃H₆— and —C₄H₈—.

In cases where one or more —CH₂— groups of the alkylene chain are replaced by a group, these may be replaced by either —O— or —CO— groups, or by a heterocyclic ring (e.g. a saturated heterocyclic ring such as a piperazinylene group), or an aryl ring (e.g. phenylene). Examples of such linkers in which one or more —CO— are present include —CO—, —CO—CH₂—, —CO—C₃H₆, —CO—C₅H₁₀—, —CO—C₆H₁₂—, and —CO—C₁₀H₂₀—. Other examples of suitable linkers in which two or more-O-groups are present include oligo- or poly-ethylene glycol groups, preferably polyethylene glycol groups containing from 1 to 4 ethylene oxide units, e.g. 2 or 4 ethylene oxide units.

In certain embodiments, the chemiluminescent agent conjugate for use in the invention is a compound of formula (III), or a pharmaceutically acceptable salt thereof:

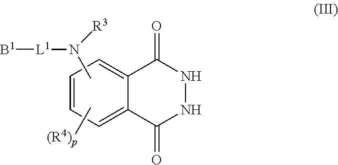

where L¹ is either a direct bond or any linker as herein described;
B¹ is any endoplasmic reticulum-targeting moiety as herein described;
R³ is hydrogen, or an alkyl group such as C₁₋₃ alkyl (e.g. methyl); and
each R⁴ is independently selected from C₁₋₆ alkyl, and —NR⁵R⁶;
R⁵ and R⁶ are independently selected from H and C₁₋₆ alkyl, preferably from H and C₁₋₃ alkyl (e.g. —CH₃); and
p is an integer from 0 to 3, preferably 0, 1 or 2, e.g. 0 or 1.

Preferred compounds of formula (III) include the following compounds of formula (IIIa) and (IIIb):

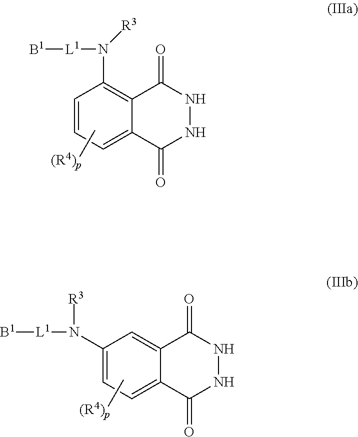

where L¹, B¹, R³, R⁴ and p are as herein defined.

In formula (III), (IIIa) and (IIIb), L¹ is preferably selected from one of the following:

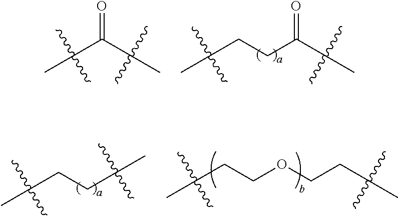

where a is an integer from 1 to 10, preferably from 3 to 10; and
b is an integer from 1 to 4, e.g. 2.

In one embodiment of formula (III), (IIIa) and (IIIb), L¹ is a group

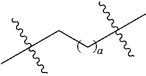

in which a is 2 or 3.

In one embodiment of formula (III), (IIIa) or (IIIb), B¹ may be one of the following groups:

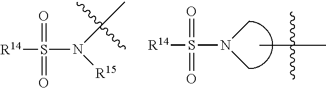

wherein
R¹⁴, R¹⁵ and

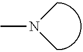

are as herein defined.

In certain embodiments, the chemiluminescent agent conjugate for use in the invention is a compound of formula (IV), or a pharmaceutically acceptable salt thereof:

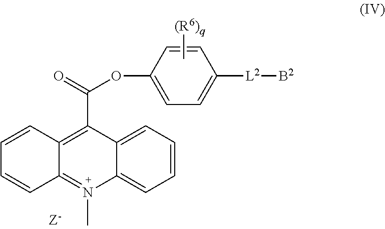

where L² is either a direct bond or any linker as herein described;
B² is any endoplasmic reticulum-targeting moiety as herein described;
each R⁶ is independently selected from halogen (e.g. F, Cl, Br, I), and C₁₋₆ alkyl (e.g. tert-butyl);
q is an integer from 0 to 4, preferably 0 or 2; and
Z is a monovalent anion, e.g. a Cl, Br, I, or CF₃OSO₂ anion.

In one embodiment of formula (IV), L² represents one of the following groups:

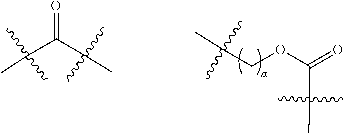

wherein a is an integer from 1 to 10, preferably 3, 4 or 5.

In one embodiment of formula (IV), L² is a group

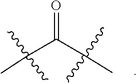 [SMILES: *C(*)=O]

In one embodiment of formula (IV), B² may be one of the following groups:

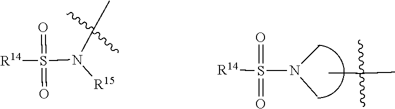

wherein
R¹⁴, R¹⁵ and

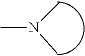

are as herein defined.

In certain embodiments, the chemiluminescent agent conjugate for use in the invention is a compound of formula (V), or a pharmaceutically acceptable salt thereof:

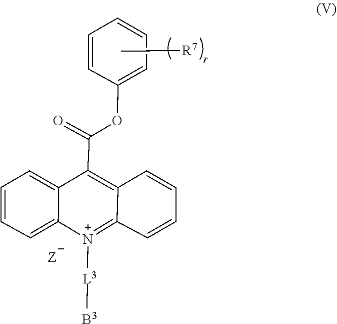

where L³ is either a direct bond or any linker as herein described;
B³ is any endoplasmic reticulum-targeting moiety as herein described;
each R⁷ is independently selected from halogen (e.g. F, Cl, Br, I), —CO₂R⁸ (where R⁸ is hydrogen or C₁₋₆ alkyl), cyano, and C₁₋₆ alkyl (e.g. tert-Bu);
r is an integer from 0 to 5, preferably 0 or 3; and
Z is a monovalent anion, e.g. a Cl, Br, I, or CF₃OSO₂ anion.

In formula (V), L³ is preferably C₁₋₁₀ alkylene, e.g. C₁₋₆ alkylene.

In one embodiment of formula (V), B³ may be one of the following groups:

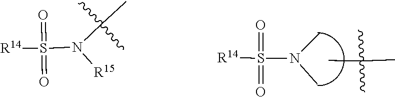

wherein
R¹⁴, R¹⁵ and

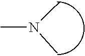

are as herein defined.

In certain embodiments, the chemiluminescent agent conjugate for use in the invention is a compound of formula (VIa), (VIb), or a pharmaceutically acceptable salt thereof:

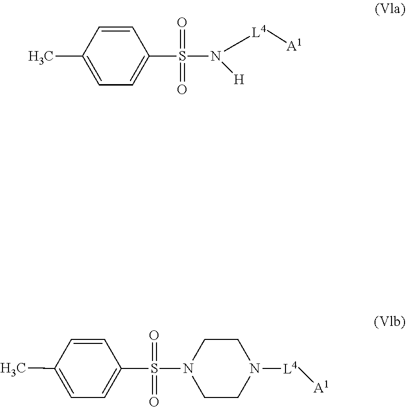

where L⁴ is either a direct bond or any linker as herein described; and
A¹ is any chemiluminescent moiety as herein described.

In formula (VI), (VIa) or (VIb), L⁴ may be selected from the following:

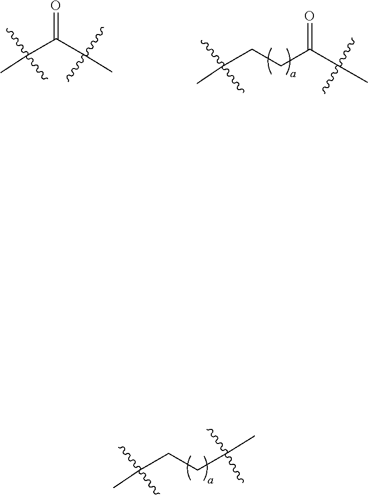

where a is an integer from 1 to 10, preferably from 3 to 10.

In one embodiment of formula (VI), (VIa) or (VIb), L⁴ is a group

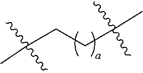

in which a is 2 or 3.

In one embodiment of formula (VI), (VIa) or (VIb), A¹ is selected from any of the following:

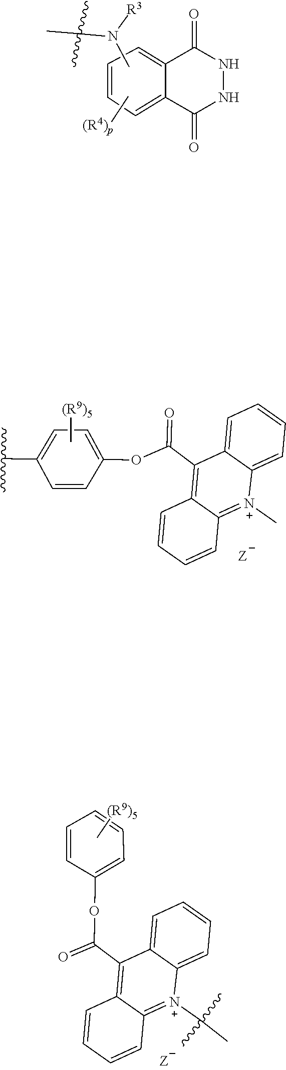

where R³ is hydrogen, or an alkyl group such as C₁₋₃ alkyl (e.g. methyl);
each R⁴ is independently selected from C₁₋₆ alkyl, and —NR⁵R⁶;
R⁵ and R⁶ are independently selected from H and C₁₋₆ alkyl, preferably from H and C₁₋₃ alkyl (e.g. —CH₃);
p is an integer from 0 to 3, preferably 0, 1 or 2, e.g. 0 or 1;
Z is a monovalent anion, e.g. a Cl, Br, I, or CF₃OSO₂ anion;
each R⁹ is independently selected from halogen (e.g. F, Cl, Br, I) and C₁₋₆ alkyl (e.g. tert-Bu); and
s is an integer from 0 to 4, preferably 0, 2 or 3.

In certain embodiments, the chemiluminescent agent conjugate for use in the invention is a compound of formula (VII), or a pharmaceutically acceptable salt thereof:

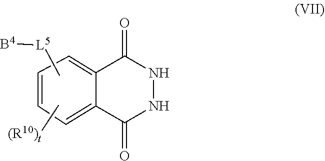

where L⁵ is either a direct bond or any linker as herein described;
B⁴ is any endoplasmic reticulum-targeting moiety as herein described;
each R¹⁰ is independently selected from C₁₋₆ alkyl (e.g. methyl), and —NR¹¹R¹²;
R¹¹ and R¹² are independently selected from H and C₁₋₆ alkyl, preferably from H and C₁₋₃ alkyl (e.g. —CH₃); and
t is an integer from 0 to 3, preferably 1 or 2.

Preferred compounds of formula (VII) include the following compounds of formula (VIIa):

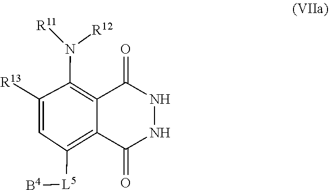

where L⁵, B⁴, R¹¹ and R¹² are as herein defined; and R¹³ is H or C₁₋₃ alkyl.

In formulae (VII) and (VIIa), L⁵ is preferably C₁₋₁₁ alkylene, more preferably C₂₋₈ alkylene, e.g. propylene.

The chemiluminescent agent conjugates herein described may be prepared using methods and procedures known in the art. Suitable methods include those described in WO 2019/243757, the entire content of which is incorporated herein by reference. Methods for the preparation of luminol derivatives for use in the invention include those described by Mikroulis et al. in J. Org. Chem. (see https://doi.org/10.1021.acs.joc.1c00890), the entire content of which is incorporated herein by reference.

Methods which may be used for covalently attaching a chemiluminescent agent to the endoplasmic reticulum-targeting moiety include known coupling techniques. The exact method used will be dependent on the exact nature of the chemiluminescent agent, the endoplasmic reticulum-targeting moiety and the linker (where present), specifically the nature of any pendant functional groups involved in forming the linkage. Where pendant functional groups are already present on the binding partners these may be employed in linking the various moieties. If necessary, one or more components of the conjugate (i.e. the chemiluminescent moiety, the linker, and the ER-targeting moiety) may be functionalised, e.g. to include reactive functional groups which may be used to couple the components. Suitable reactive groups include carboxylic acid, hydroxy, thiol, carbonyl, acid halide, primary and secondary amines, aryl halides and pseudohalides, alkyl halides and pseudohalides, alkenyl halides and pseudohalides, terminal alkynes, clickable moieties, etc. Methods for the introduction of such functional groups are well known in the art.

Examples of methods which may be used to covalently link the chemiluminescent agent to one or more ER-targeting moieties include, but are not limited to, the following: amide bond formation, ether bond formation, ester bond formation, thioester bond formation, cross-coupling reactions, olefin metathesis reactions, electrophilic aromatic substitutions, click chemistry, nucleophilic substitution reactions, etc.

Compounds for use as starting materials in the preparation of the conjugates herein described are either known from the literature or may be commercially available.

Alternatively, these may readily be obtained by methods known from the literature. A more detailed description of how to prepare the compounds for use in accordance with the invention is found in the Examples.

The chemiluminescent agent conjugates as herein described are in themselves novel and form a further aspect of the invention. Methods for their preparation comprising the step of linking one or more chemiluminescent agents to one or more ER-targeting moieties, for example using any of the techniques herein described, form a further aspect of the invention.

For use in PDT, the ER-targeted chemiluminescent agents herein described are used in combination with a photosensitizer or a precursor of a photosensitizer. Key to the invention is that these should come into close proximity to one another in the cell ER in order that the chemiluminescent agent can ‘activate’ the photosensitizing agent. These agents may be provided individually for separate, simultaneous or sequential administration to the patient in a method of PDT. Alternatively, these may be provided in a single formulation in which both the ER-targeted chemiluminescent agent and the photosensitizer (or precursor) are present. Such formulations form part of the invention.

For use in the invention, any photosensitizer (or photosensitizer precursor) should be capable of accumulating in the endoplasmic reticulum of the target cells (or translocating to the ER) following its in vivo administration to ensure that this comes into close proximity to the chemiluminescent compound. For example, this may be an ER-localising photosensitizer or precursor thereof.

Examples of photosensitizing agents and precursors which are capable of targeting the endoplasmic reticulum include: 5-ALA and its derivatives (following translocation from the mitochondria), mTHPC, temoporfin, chlorin e6, phthalocyanines, anthraquinones and derivatives thereof (e.g. hypericin, hypocrellins [A, B], cercosporin, calphostin, elsinochromes [A, B, C]), and their pharmaceutically acceptable salts. Other ER-accumulating photosensitizing agents are known in the art and these may also be used in the invention.

Other known photosensitizers and precursors may be used in the invention subject to appropriate modification to confer the desired targeting properties. For example, these may be encapsulated within a suitable nanocarrier which has ER-targeting ability. In these embodiments, a wider range of photosensitizers may be used and it is envisaged that any known photosensitizer (or precursor) suitable for use in PDT may be employed. A range of suitable agents are known in the art and include, for example, 5-aminolevulinic acid (5-ALA) and derivatives of 5-ALA (leading to production of protoporphyrin IX); porphyrins; phthalocyanines such as metallated phthalocyanines which may optionally be sulphonated (e.g. AlPcS), e.g. di-sulphonated aluminium phthalocyanines such as AlPcS₂ or AlPcS₂ₐ, or aluminium phthalocyanine tetra-sulfonate (A1PcS₄); sulphonated tetraphenylporphyrins (e.g. TPPS₂ₐ, TPPS₄, TPPS₁ and TPPS₂₀); chlorins such as tetra (m-hydroxyphenyl)chlorins (m-THPC) (e.g. temoporfin which is marketed under the tradename Foscan); chlorin derivatives including bacteriochlorins and ketochlorins; mono-L-aspartyl chlorin e6 (NPe6) or chlorin e6; natural and synthetic porpyhrins including hematoporphyrin and benzoporphyrins; anthraquinones and derivatives thereof (e.g. hypericin, hypocrellins [A, B], cercosporin, calphostin, elsinochromes [A, B, C]).

Pharmaceutically acceptable salts of any of these photosensitizers (or precursors) may also be used. Such salts include salts with pharmaceutically acceptable organic or inorganic acids or bases.

Derivatives of 5-ALA which may be used in the invention include any derivative of 5-ALA capable of forming PpIX in vivo. Typically, such derivatives will be precursors of PpIX in the biosynthetic pathway for heme and which are therefore capable of producing PpIX at the target site following administration. Suitable precursors of PpIX include 5-ALA prodrugs such as 5-ALA esters.

The following are amongst the preferred photosensitizers and precursors for use in the invention: 5-ALA, mTHPC, temoporfin, chlorin e6, sulphonated aluminium phthalocyanines, anthraquinones and derivatives thereof (e.g. hypericin, hypocrellins [A, B], cercosporin, calphostin, elsinochromes [A, B, C]), and their pharmaceutically acceptable salts. Particularly preferred for use in the invention is cercosporin, 5-ALA and its pharmaceutically acceptable derivatives (e.g. pharmaceutically acceptable salts, or methyl or hexyl esters).

The particular choice of chemiluminescent moiety will be dependent on various factors, including the nature of the tumour to be treated, but can readily be selected by those skilled in the art. As will be understood, the choice of chemiluminescent agent will also be dependent on the photosensitizer to be used in the PDT treatment since the wavelength of light it emits should be suitable for photoactivation of the photosensitizer either by direct light absorbance or energy transfer mechanisms.

Examples of suitable chemiluminescent agent-photosensitizer “pairs” may readily be determined by those skilled in the art. The following are provided by way suitable non-limiting examples. Where the photosensitizer is PpIX (e.g. produced in vivo following administration of 5-ALA), luminol or isoluminol may be used as the chemiluminescent agent. The photosensitizer hypericin is particularly suitable for use with the chemiluminescent agent lucigenin since these moieties can form pi-stacks for very efficient intramolecular energy transfer especially in the presence of binding agents such as the metal chelators DTPA or EDTA. Luminol and mTHPC represent a very efficient energy transfer pair. Other efficient energy transfer pairs include luminol-erythrosine B, luminol-hypocrellins, luminol-cercosporin, luminol-calphostin, luminol-elsinochromes acridine esters-hypocrellins, lucigenin-hypocrellins, acridine esters-cercosporin, lucigenin-cercosporin, acridine esters-hypericin, and lucigenin-hypericin. Haematoporphyrin derivative (HPD) or sulphonated aluminium phthalocyanine may be used with either luminol or lucigenin. However, these are only indicative examples of potential functional pairs and others can readily be determined by those skilled in the art.

The ER-targeted chemiluminescent agents herein described are intended for use in methods of photodynamic therapy and are suitable for use in the treatment of disorders or abnormalities of cells or tissues within the body which are responsive to photodynamic therapy. Such methods will involve the simultaneous, separate or sequential use of a photosensitizer or a precursor of a photosensitizer as herein described.

In general, cells which are metabolically active are responsive to photodynamic treatment. Examples of metabolically active cells are those which undergo abnormal growth, such as an increased number of cells/increased cell proliferation, abnormal maturation and differentiation of cells, or abnormal proliferation of cells. Any condition characterised by such a growth pattern may be treated in accordance with the PDT methods herein described.

Disorders or abnormalities which may be treated include, but are not limited to, malignant and pre-malignant cancer conditions, such as cancerous growths or tumours, and their metastases; tumours such as sarcomas and carcinomas, in particular solid tumours. The invention is particularly suitable for the treatment of tumours, especially those which are located below the surface of the skin, i.e. internal cancers or deeply-sited cancers.

PDT in accordance with the invention may be applied in two ways: (i) as a treatment for malignant or pre-malignant conditions (e.g. gliomas) without the need for an external light source as in classical PDT; or (ii) as a repeatable, adjuvant, post-operative, photochemical treatment to subdue any active neoplastic foci left behind, which could lead to either recurrence or disease dissemination. The treatment may be effectively used to manage and contain the condition (e.g. brain cancer) for life through repeated treatment sessions. Treatment of occult metastasis of the primary disease may also be carried out without the need for previous diagnosis.

Examples of tumours that may be treated using the invention are sarcomas, including osteogenic and soft tissue sarcomas; carcinomas, e.g. breast, lung, cerebral, bladder, thyroid, prostate, colon, rectum, pancreas, stomach, liver, uterine, hepatic, renal, prostate, cervical and ovarian carcinomas; lymphomas, including Hodgkin and non-Hodgkin lymphomas; neuroblastoma, melanoma, myeloma, Wilm's tumour; leukemias, including acute lymphoblastic leukaemia and acute myeloblastic leukaemia; astrocytomas, gliomas and retinoblastomas; mesothelioma. However, the invention finds particular value in the treatment of deep lying cancerous lesions that are difficult to access non-invasively. Treatment of gliomas (e.g. GMB) forms a preferred aspect of the invention.

Other examples of metabolically active cells are inflamed cells. Inflammatory diseases such as rheumatoid arthritis may thus also be treated using the PDT methods in accordance with the invention.

For use in any of the PDT methods herein described, the ER-targeted chemiluminescent agent will generally be provided in a pharmaceutical composition with at least one pharmaceutically acceptable carrier or excipient. Such compositions form a further aspect of the invention. These may also comprise the selected photosensitizer (or precursor), although it is envisaged that in most cases the photosensitizer (or its precursor) will be provided in a different formulation for separate administration to the patient.

The pharmaceutical compositions as herein described may be formulated using techniques well known in the art. The route of administration will depend on the intended use and, in particular, the location of the cells or tissues to be treated. Typically, these will be administered systemically and may thus be provided in a form adapted for parenteral administration, e.g. by intradermal, subcutaneous, intraperitoneal, intravenous or intratumoural injection, or by infusion via a drip. Suitable pharmaceutical forms include suspensions and solutions which contain the conjugate and/or the photosensitizer (or its precursor) together with one or more inert carriers or excipients. Suitable carriers include saline, sterile water, phosphate buffered saline and mixtures thereof. Preferably, the compositions will be used in the form of an aqueous suspension or solution in water or saline, e.g. phosphate-buffered saline.

The compositions may additionally include other agents such as emulsifiers, suspending agents, dispersing agents, viscosity modifiers, solubilising agents, stabilisers, buffering agents, preserving agents, etc. The compositions may be sterilised by conventional sterilisation techniques.

In one embodiment the ER-targeted chemiluminescent agent is provided in the form of a solution in water or in saline (or in any other pharmaceutically relevant, biocompatible vehicle) which is suitable for injection intravenously or intratumourally. This may be administered either as a single dose or in repeated doses.

In one embodiment the chemiluminescent agent conjugate may be administered in the form of a slow-release formulation. Suitable delayed release formulations are known in the art and include any formulation which is capable of the continuous slow release of the agent in vivo. One example of a suitable delayed release formulation is an injectable implant which provides for sustained release in vivo. Such an implant may be an in situ forming implant based on biocompatible and biodegradable polymers containing nanoparticles of the active compounds. These will provide sustained delivery of the chemiluminescent agent to assure prolonged luminescence, thereby achieving optimised therapeutic effect of the treatment.

In another aspect, the chemiluminescent conjugate may be provided in the form of thermoresponsive formulations which become thermogels at physiological temperature (i.e. once delivered to the body). These can be formulated to optimally release their load over a period of up to 15 hours, e.g. 10 to 15 hours. The use of temperature-responsive polymers allows the formulation of low viscosity solutions which are suitable for subcutaneous injection and which, in response to body temperature, undergo an in situ phase-transition and turn into a gel. To optimise the thermosetting properties of the gel-network different polymers and their copolymers may be used. Such polymer materials are known and used in the art and include, for example: poly(lactic-co-glycolic acid) (PLGA); alginate/hyaluronic acid; poly(N-isopropylacrylamide); and poloxamers.

Nanoparticles and/or microparticles containing the chemiluminescent conjugates may also be provided in order to provide a controlled and continuous release of the active over a prolonged period, e.g. over 10 to 15 hours. Examples of such carriers include (i) micellar carriers, (ii) liposomes, (iii) dendrimeric or polymeric nanocarriers, and (iv) solid lipid nanoparticles. Any such particles may be included in the thermoresponsive formulations herein described such that these form a reservoir for release of the active in the in situ produced gel network.

The compositions herein described may be administered systemically (e.g. orally or parenterally), or alternatively these may be locally applied (e.g. topically) at or near the affected site. The route of administration will depend on the severity, nature and location of the disease to be treated as well as the photosensitizer (or precursor) used. Compositions that may be administered systemically include plain or coated tablets, capsules, suspensions and solutions. Compositions that may be administered locally (e.g. topically) include gels, creams, ointments, sprays, lotions, and any of the other conventional pharmaceutical forms in the art. Creams, ointments and gels may be formulated with an aqueous or oily base with the addition of suitable thickening and/or gelling agents.

Typically, the methods herein described might involve the initial step of administration of an effective amount of a composition which contains the photosensitizer, e.g. by intravenous injection. The photosensitizer (or precursor) is then allowed to distribute to the desired target area of the body and to allow for in situ generation of the active photosensitizing agent, e.g. PpIX, prior to administration of the ER-targeted chemiluminescent agent, e.g. intravenously either by injection and/or a drip. The time profile for PpIX generation in cells following 5-ALA administration can be several hours (typically this might peak between 2-10 hours after administration), hence it is desirable to delay the delivery of the ER-targeted chemiluminescent agent. This can either be achieved by delaying its administration or by means of delayed release formulations as herein described. Whilst it is envisaged that administration of the photosensitizer will typically take place prior to administration of the ER-targeted chemiluminescent agent, delivery of these may nevertheless be simultaneous, for example, where the ER-targeted chemiluminescent agent is provided in the form of a delayed release formulation (e.g. in the form of any of the nanoparticulate and/or microparticulate carrier systems as herein described).

For example, the patient may first have a 5-ALA injection and then at the right timeframe and within the appropriate therapeutic window, will receive either a second injection of the ER-targeted chemiluminescent agent or will be placed on a drip containing this agent for as long as required. This set-up is minimal in demands subject to any post-treatment monitoring.

The effective dose of the compositions herein described, the number of doses, and precise timing for administration will depend on various factors, including the nature of the ER-targeted chemiluminescent agent, the photosensitizer (or precursor), their mode(s) of administration, the condition to be treated, the patient, etc., and may be adjusted accordingly.

A further aspect of the invention relates to a method of photodynamic therapy of cells or tissues of a patient, said method comprising the step of administering to said cells or tissues:
(a) an effective amount of an endoplasmic reticulum-targeted chemiluminescent agent as herein described and, simultaneously, separately, or sequentially thereto, an effective amount of a photosensitizer or photosensitizer precursor; or
(b) an effective amount of a pharmaceutical composition which comprises an endoplasmic reticulum-targeted chemiluminescent agent as herein described and a photosensitizer or photosensitizer precursor.

In a further aspect the invention provides a product comprising an endoplasmic reticulum-targeted chemiluminescent agent as herein described, and a photosensitizer or precursor for simultaneous, separate or sequential use in a method of photodynamic therapy, e.g. in any of the PDT methods herein described.

In a still further aspect the invention provides a kit comprising: (i) an endoplasmic reticulum-targeted chemiluminescent agent as herein described; and separately (ii) a photosensitizer or photosensitizer precursor; and optionally (iii) instructions for the use of (i) and (ii) in a method of photodynamic therapy. When used, the active components of the kit (i.e. (i) and (ii)) may be administered simultaneously, separately or sequentially.

The invention will now be described further with reference to the following non-limiting Examples and the accompanying drawings in which:

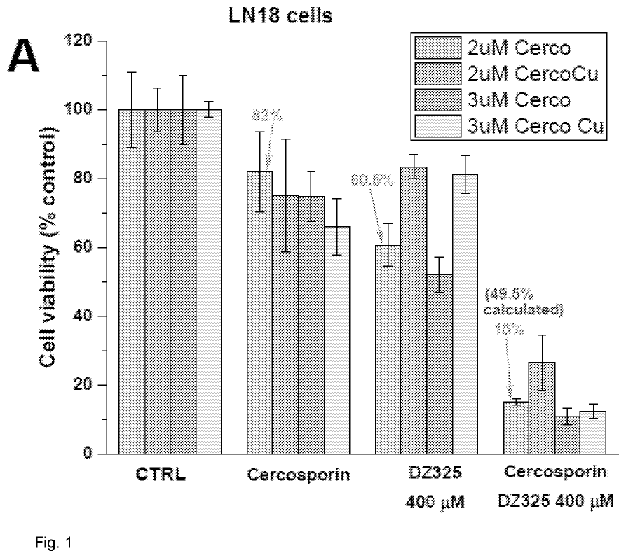

FIG. 1—Cell viability of LN18 cells: Compound DZ325+cercosporin.

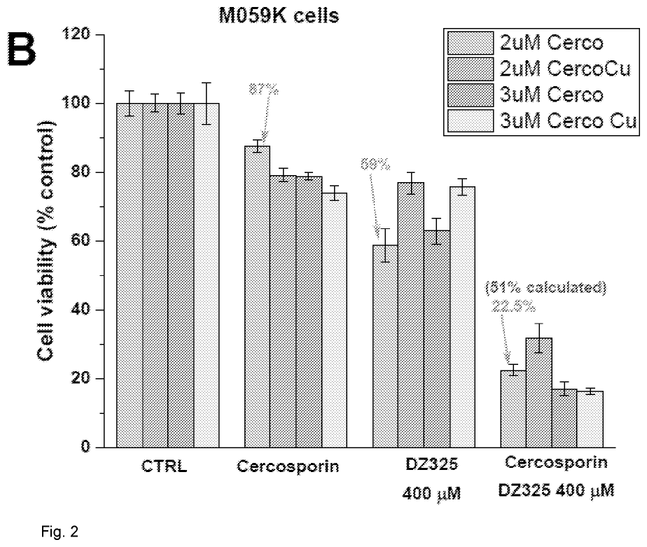

FIG. 2—Cell viability of M059K cells: Compound DZ325+cercosporin.

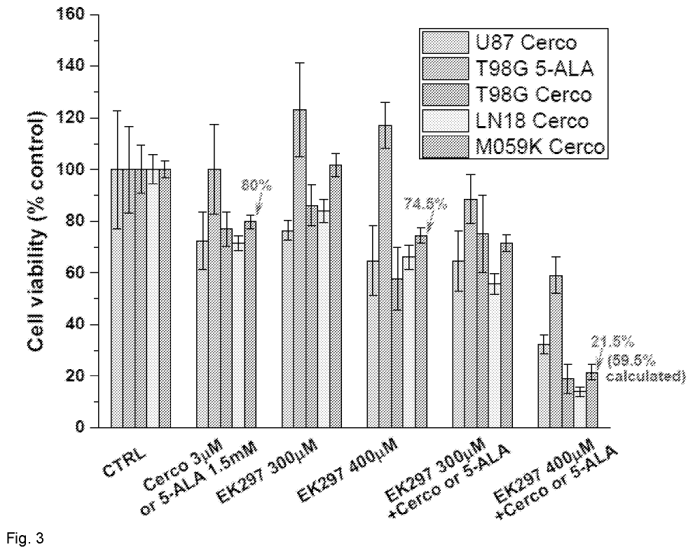

FIG. 3—Cell viability of U87, T98G, LN18 and M059K cells: Compound EK297+cercosporin or 5-ALA.

### Examples

### Example 1—Preparation of N-(3-((1,4-dioxo-1,2,3,4-tetrahydrophthalazin-5-yl)amino)propyl)-4-methylbenzenesulfonamide (DZ325)

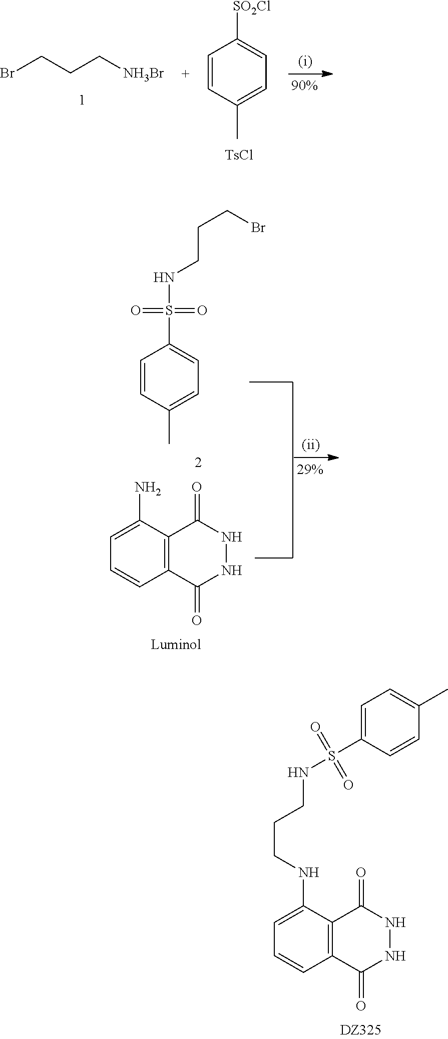 [0206] Scheme 1—Synthesis of sulfonamide-alkyl functionalized luminol derivative. Reagents and conditions: (i) Et₃N, DCM, (ii) NMP, 120° C.

### Experimental Procedure

### Synthesis of N-(3-bromopropyl)-4-methylbenzenesulfonamide 2

Triethylamine (437 mg, 4.32 mmol) was added dropwise to a cooled (0° C.) stirred suspension of 4-toluenesulfonylchloride (342 mg, 1.79 mmol) and 3-bromopropylamine hydrobromide (453 mg, 2.07 mmol) in dry dichloromethane (10 mL). The resulting mixture was stirred at that temperature for 15 minutes, then dichloromethane (50 mL) was added, washed with 2N HCl (2×40 mL) and brine (40 mL), dried (Na₂SO₄) and the solvent was evaporated, affording 2 (471 mg, 90%).

¹H NMR (200 MHz, CDCl₃) δ: 7.76 (d, J=8.3 Hz, 1H, ArH-oS), 7.31 (d, J=8.5 Hz, 1H, ArH-oMe), 5.01 (bs, 2H, NH), 3.41 (t, J=6.3 Hz, 1H, CH₂Br), 3.09 (t, J=6.0 Hz, 1H, CH₂N), 2.43 (s, 3H, CH₃), 2.01 (qui, J=6.4 Hz, 2H, CH₂).

### Synthesis of N-(3-((1,4-dioxo-1,2,3,4-tetrahydrophthalazin-5-yl)amino)propyl)-4-methylbenzenesulfonamide DZ325

A solution of luminol (313 mg, 1.77 mmol) and bromide 2 (514 mg, 1.76 mmol) in N-methylpyrrolidone (2 mL) was stirred at 120° C. for 2 days. After cooling, the mixture was poured into stirred ice-water (60 mL) and stirred for 15 min. The precipitate formed was filtered, washed with water (3×5 mL) and dried in vacuo. The residue was dissolved in 5% THF/dichloromethane and purified via column chromatography (5-10% THF/dichloromethane). The crude DZ325 obtained was triturated with diethyl ether and hexane, affording DZ325 as a pale yellow powder (200 mg, 29%).

¹H NMR (400 MHz, DMSO-d₆) δ: 11.39 (bs, 1H, CONH), 11.14 (bs, 1H, CONH), 8.97 (bs, 1H, ArNH), 7.66 (app d, J=8.3 Hz, 2H, H-2′), 7.59 (t, J=5.8 Hz, 1H, SNH), 7.55 (t, J=8.1 Hz, 1H, H-7), 7.35 (app dd, J=8.5, 0.6 Hz, 2H, H-3′), 6.96 (d, J=7.3 Hz, 1H, H-8), 6.76 (dd, J=8.3, 0.5 Hz, 1H, H-6), 3.16 (q, J=6.5 Hz, 2H, ArNCH₂), 2.85 (q, J=6.8 Hz, 2H, SNCH₂), 2.35 (s, 3H, CH₃), 1.67 (qui, J=6.8 Hz, 2H, CH₂).

¹³C NMR (50 MHz, DMSO-d₆) δ: 161.61, 151.38, 149.90, 142.57, 137.53, 134.51, 129.62, 126.60, 126.53, 111.12, 110.89, 108.72, 28.28, 20.98, 20.96.

ES-MS m/z for C₁₈H₁₉N₄O₄S [M−H]⁻: calcd. 387.1, found 387.1.

### Example 2—Preparation of 9-((2,6-dibromo-4-(4-tosylpiperazine-1-carbonyl)phenoxy)carbonyl)-10-methylacridin-10-ium trifluoromethanesulfonate (EK297)

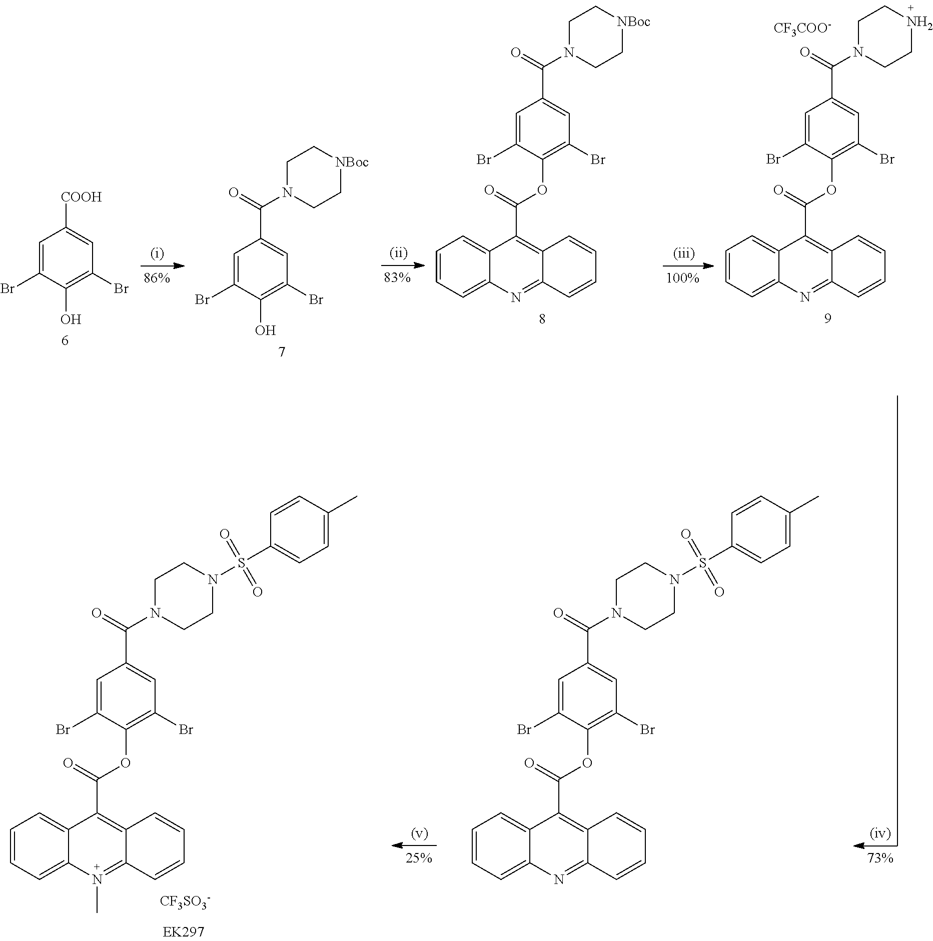 [0214] Scheme 2—Synthesis of sulphonamide functionalized acridinium ester. Reagents and conditions:
   (i) 1-Boc-piperazine, EDC, HOBt, THF, (ii) a) 9-acridinecarboxylic acid, SOCl₂, Δ, b) Et₃N, Py, DCM, (iii) TFA, CHCl₃, (iv) TsCl, DIPEA, DMF, (v) MeOTf, DCM.

### Experimental Procedure

### Synthesis of tert-butyl 4-(3,5-dibromo-4-hydroxybenzoyl) piperazine-1-carboxylate 7

HOBt (259 mg, 1.69 mmol) and EDC (787 mg, 5.07 mmol) were successively added in a solution of 3,5-dibromo-4-hydroxybenzoic acid (1 g, 3.38 mmol) and 1-Boc-piperazine (1.89 g, 10.14 mmol) in dry tetrahydrofuran (40 mL) and the resulting mixture was stirred under argon for 7.5 hours. The mixture was decanted into ice/water, acidified with 1N HCl and the precipitate was filtered, washed with water and dried, affording phenol 7 as a white solid (1.35 g, 86%).

¹H NMR (200 MHz, CDCl₃) δ: 7.53 (s, 2H, ArH), 6.32 (bs, 1H, OH), 3.65-3.40 (m, 8H, piperazine), 1.47 (s, 9H, tBu). ¹³C NMR (50 MHz, CDCl₃) δ: 167.90, 154.51, 151.52, 131.10, 129.10, 111.04, 80.64, 47.67 (br), 43.61 (br), 28.36.

ES-HRMS m/z for C₁₆H₂₀Br₂N₂NaO₄ [M+Na]⁺: calcd. 486.9667, found 486.9661.

### Synthesis of 2,6-dibromo-4-(4-(tert-butoxycarbonyl)piperazine-1-carbonyl)phenyl acridine-9-carboxylate 8

A suspension of 9-acridinecarboxylic acid hydrate (289 mg, 1.29 mmol) in thionyl chloride (3 mL) was stirred at reflux under argon for 5 hours. After cooling, excess thionyl chloride was removed under high vacuum and dry dichloromethane (7 mL) was added under argon. To the resulting solution were added successively phenol 7 (600 mg, 1.29 mmol), triethylamine (0.36 mL, 2.58 mmol) and pyridine (0.052 mL, 0.65 mmol) and the whole was stirred for 18 hours. Then, dichloromethane (30 mL) was added, the phases were separated and the aqueous phase was washed with dichloromethane (2×30 mL). The organic phases were combined, washed successively with 1N HCl (2×20 mL), aq. NaHCO₃ (20 mL) and brine (20 mL), dried (Na₂SO₄) and the solvent was evaporated. Column chromatography (10% MeCN/dichloromethane) of the residue afforded 8 as a yellow solid (715 mg, 83%).

¹H NMR (200 MHz, CDCl₃) δ: 8.74 (d, J=8.8 Hz, 2H, H-4), 8.34 (d, J=8.8 Hz, 2H, H-1), 7.87 (ddd, J=8.8, 6.6, 1.4 Hz, 2H, H-3), 7.76 (s, 2H, H-3′), 7.69 (ddd, J=8.8, 6.7, 1.3 Hz, 2H, H-2), 3.80-3.40 (m, 8H, piperazine), 1.49 (s, 9H, (Bu).

¹³C NMR (50 MHz, CDCl₃) δ: 166.53, 163.69, 154.43, 148.68, 147.62, 136.32, 133.22, 131.61, 130.36, 130.14, 127.69, 125.71, 122.92, 118.29, 80.58, 47.69 (br), 43.59 (br), 43.31 (br), 42.45 (br), 28.37.

ES-HRMS m/z for C₃₀H₂₈Br₂N₃O₅ [M+H]⁺: calcd. 667.0375, found 670.0370.

### Synthesis of 4-(4-((acridine-9-carbonyl)oxy)-3,5-dibromobenzoyl)piperazin-1-ium 2,2,2-trifluoroacetate 9

Acridine 8 (162 mg, 0.24 mmol) was added into a mixture of trifluoroacetic acid (2 mL) and chloroform (2 mL) and the solution was stirred for 4 hours. Evaporation of the mixture left salt 9 as white solid (165 mg, quantitative).

¹H NMR (200 MHz, MeOD-d₄) δ: 8.84 (d, J=8.9 Hz, 2H, H-4), 8.36 (d, J=8.9 Hz, 2H, H-1), 8.12 (ddd, J=8.8, 6.7, 1.2 Hz, 2H, H-3), 8.02 (s, 2H, H-3′), 7.89 (ddd, J=8.5, 6.7, 1.1 Hz, 2H, H-2), 4.03-3.83 (m, 4H, piperazine), 3.46-3.33 (m, 4H, piperazine).

¹⁹F NMR (188 MHz, MeOD-d₄) δ: −77.99. ES-HRMS m/z for C₂₅H₂₀Br₂N₃O₃ [M]⁺: calcd. 569.9845, found 569.9899.

### Synthesis of 2,6-dibromo-4-(4-tosylpiperazine-1-carbonyl)phenyl acridine-9-carboxylate 10

Tosyl chloride (95 mg, 0.5 mmol) was added to a stirring solution of 9 (310 mg, 0.45mmol) and DIPEA (0.25 mL, 1.41 mmol) in DMF (8 mL) at 0° C. under argon and the reaction mixture was left stirring for 3 hours. Water (30 mL) was added, the precipitate was filtered, washed with water, and dried in vacuo, leaving the desired product 10 as a yellow solid (235 mg, 73%).

¹H NMR (200 MHz, CDCl₃) δ: 8.70 (d, J=8.8 Hz, 2H, H-4), 8.32 (d, J=8.7 Hz, 2H, H-1), 7.84 (ddd, J=8.7, 6.7, 1.1 Hz, 2H, H-3), 7.71-7.60 (m, 6H, H-2,3′,2_{TOS}), 7.35 (app d, J=8.1 Hz, 2H, H-3_{TOS}), 3.74 (bs, 4H, piperazine), 3.05 (bs, 4H, piperazine), 2.44 (s, 3H, CH₃).

¹³C NMR (50 MHz, CDCl₃) δ: 166.47, 163.76, 148.79, 147.90, 144.42, 135.81, 133.25, 132.26, 131.71, 130.47, 130.26, 130.09, 127.85, 127.82, 125.76, 123.02, 118.42, 46.05, 21.70.

ES-HRMS m/z for C₃₂H₂₆Br₂N₃O₅S [M+H]⁺: calcd. 723.9939, found 723.9998.

### Synthesis of 9-((2,6-dibromo-4-(4-tosylpiperazine-1-carbonyl)phenoxy)carbonyl)-10-methylacridin-10-ium trifluoromethanesulfonate EK297

Methyl triflate (36 mg, 0.22 mmol) was added to a solution of acridinic ester 10 (160 mg, 0.22 mmol) in dry dichloromethane (7 mL) and stirred for 24 hours under argon. The volatiles were evaporated and the residue was washed (sonicate/centrifuge) with cold EtOAc (4×5 mL), precipitated with DCM/Toluene and washed with hexane (x2), leaving acridinium ester EK297 as a yellow powder (48 mg, 25%).

¹H NMR (200 MHz, CD₃CN) δ: 8.98 (d, J=9.0 Hz, 2H, H-4), 8.72 (d, J=9.1 Hz, 2H, H-1), 8.50 (ddd, J=9.4, 6.9, 1.3 Hz, 2H, H-3), 8.14 (dd, J=8.7, 6.7 Hz, 2H, H-2), 7.78 (s, 2H, H-3′), 7.64 (app d, J=8.3 Hz, 2H, H-2_{TOS}), 7.43 (d, J=8.2 Hz, 2H, H-3_{TOS}), 4.90 (s, 3H, NCH₃), 3.95-3.27 (m, 4H, piperazine), 3.00 (app bs, 4H, piperazine), 2.44 (s, 3H, CH₃). ¹³C NMR (50 MHz, CD₃CN) δ: 166.59, 162.47, 146.98, 145.37, 143.24, 140.59, 138.75, 133.49, 132.88, 130.85, 130.64, 129.15, 128.75, 124.42, 120.31, 118.27, 46.72, 40.96, 21.56. ¹⁹F NMR (188 MHz, CD₃CN) δ: −78.65.

ES-HRMS m/z for C₃₃H₂₈Br₂N₃O₅S [M]⁺: calcd. 738.0100, found 738.0099.

### Example 3—Cercosporin or 5-ALA+DZ325 or EK297 in GBM Cell Lines

The efficacy of the chemiluminescent compounds DZ325 and EK297 was tested in cell cultures of human glioblastoma multiforme cell lines (U87, T98G, M059K and LN-18).

The cells were inoculated in 96-well plates and left to attach overnight at 37° C. in a 5% CO₂, humidified atmosphere. Subsequently the cells were incubated with (i) the photosensitizer (cercosporin) or the photosensitizer precursor (5-ALA, for the generation of the photosensitizer PpIX); and (ii) the compound at the appropriate concentration. In the case of 5-ALA, its introduction to the cell culture preceded that of the chemiluminescent compound by 1 hour to ensure adequate PpIX production. In the case of cercosporin, it was administered at the same time as the chemiluminescent compound.

The chemiluminescent compounds were prepared as 50 mM stock solutions in DMSO (or 1-methyl-2-pyrrolidone) and diluted in cell media to their final concentrations. Cercosporin was prepared as a 4 mM solution in DMSO and appropriately diluted in cell media. 5-ALA was made to its final concentration in Optimem. In the case of the luminol-based compound (DZ325), Cu was in some cases added to the cell media in the form of CuSO₄ at a final concentration of 100 μM.

24 hours from the introduction of the chemiluminescent compounds to the cells, the cell media was removed and replaced with 100 μL of media containing 0.5 mg/mL thiazolyl blue salts for the performance of a standard MTT viability assay. The MTT containing medium was left to incubate for 1 to 3 hours and then removed and replaced with 100 μL DMSO. The endpoint absorbance measurement was made at 561 nm. Media only controls were used as 100% cell viability controls. The experimental cell viability of the combined treatment (photosensitizer+chemiluminescent compound) was compared to the theoretical additive treatment of the photosensitizer+chemiluminescent compound which was calculated as follows:

$$\\ \frac{\begin{matrix}cell viability ( photosensitizer only ) \% \times \\ cell viability ( chemiluminescent moiety only ) \%\end{matrix}}{100 \% ( control )}$$

A significantly higher viability of the combined treatment (experimental) than the theoretically calculated additive value is considered evidence of a synergistic effect between the chemiluminescent compound and the photosensitizer.

The results are presented in FIGS. 1 to 3. In each figure, one example of synergy is highlighted. For example, in FIG. 3, 3 μM cercosporin alone reduced the cell viability to 80%, while 400 μM EK297 reduced the toxicity to 74.5%. Calculated cell viability is determined according to the following equation (in which “PS”=photosensitizer; “CL”=chemiluminescent agent):

$$\\ Calculated cell viability PS + CL ( \% ) = \frac{Cell viability PS ( \% ) \times Cell viability CL ( \% )}{Media control cell viability ( 100 \% )}$$

The calculated cell viability for 3 μM cercosporin+400 μM EK297 is 59.5%, however the experimental value of the corresponding cell viability was found to be 21.5%. This suggests a cytotoxic synergy between EK297 and cercosporin. The examples of synergy shown in FIGS. 1 and 2 when using DZ325 are calculated in the same way.

1. An endoplasmic reticulum-targeted chemiluminescent agent for use in a method of photodynamic therapy.

2. An agent for use as claimed in claim 1, wherein said agent is a conjugate comprising at least one chemiluminescent moiety attached to or otherwise associated with at least one endoplasmic reticulum-targeting moiety.

3. An agent for use as claimed in claim 2, wherein said conjugate is a compound of general formula (I), or a pharmaceutically acceptable salt thereof: 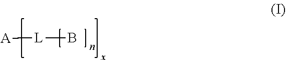
in which A represents a chemiluminescent moiety;
each L, which may be the same or different, is either a direct bond or a linker (e.g. an organic linker);
each B, which may be the same or different, represents an endoplasmic reticulum-targeting moiety;
n is an integer from 1 to 3, preferably 1; and
x is an integer from 1 to 3, preferably 1.

4. An agent for use as claimed in claim 3, wherein said conjugate is a compound of formula (II), or a pharmaceutically acceptable salt thereof:
A-L-B (II)
in which A, L and B are as defined in claim 3.

5. An agent for use as claimed in any one of the preceding claims, wherein said chemiluminescent agent or chemiluminescent moiety is selected from the group consisting of luminol, isoluminol, lucigenin, acridinium esters, oxalate esters, and derivatives thereof.

6. An agent for use as claimed in claim 5, wherein said chemiluminescent agent or chemiluminescent moiety is luminol, isoluminol, an acridinium ester, or a derivative thereof.

7. An agent for use as claimed in any one of claims 2 to 6, wherein said endoplasmic reticulum-targeting moiety is a sulphonamide or sulphonamide derivative.

8. An agent for use as claimed in claim 7, wherein said endoplasmic reticulum-targeting moiety is a group having one of the following structures: 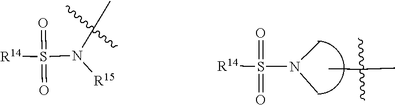
wherein
R¹⁴ is C₁₋₆ alkyl or an optionally substituted aryl group;
R¹⁵ is hydrogen or C₁₋₆ alkyl; and 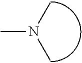
denotes an optionally substituted, nitrogen-containing heterocyclic ring.

9. An agent for use as claimed in claim 8, wherein said endoplasmic reticulum-targeting moiety is a group having the following structure: 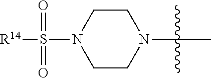
wherein
R¹⁴ is as defined in claim 8.

10. An agent for use as claimed in claim 8, wherein said endoplasmic reticulum-targeting moiety is a group having one of the following structures: 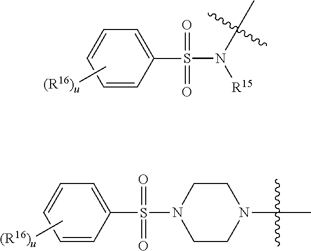
wherein
R¹⁵ is as defined in claim 8;
each R₁₆ independently represents C₁₋₃ alkyl (e.g. methyl), C₁₋₃ alkoxy (e.g. methoxy) or a halogen atom (e.g. Cl or Br); and
u is an integer from 0 to 5, preferably 1 to 3, e.g. 1.

11. An agent for use as claimed in any one of claims 3 to 10, wherein said linker L comprises an alkylene chain (preferably a C₁₋₁₅ alkylene, e.g. a C₂₋₁₁ alkylene) optionally substituted by one or more groups selected from C₁₋₃ alkyl, —O(C₁₋₃)alkyl, —OH, cycloalkyl and aryl groups; and in which one or more —CH₂— groups of the alkylene chain may be replaced by a group independently selected from —O—, —CO—, —NR— (where R is H or C₁₋₆ alkyl, preferably C₁₋₃ alkyl, e.g. methyl), cycloalkyl, heterocyclic, aryl and heteroaryl groups.

12. An agent for use as claimed in claim 11, wherein said linker L is selected from the group consisting of: —C₃H₆—, —C₄H₈—, —C₆H₁₂—, —C₈H₁₆—, —C₁₀H₂₀—, —C₁₁H₂₂—, —CO—, —CO—CH₂—, —CO—C₃H₆, —CO—C₅H₁₀—, —CO—C₆H₁₂—, —CO—C₁₀H₂₀—, and polyethylene glycol groups containing from 1 to 4 ethylene oxide units.

13. An agent for use as claimed in any one of the preceding claims which is a compound of formula (III), or a pharmaceutically acceptable salt thereof: 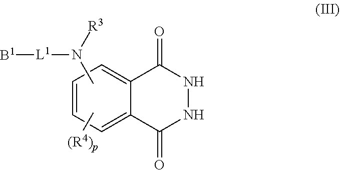
(where L¹ is a direct bond or a linker, e.g. a linker as defined in claim 11 or 12;
B¹ is an endoplasmic reticulum-targeting moiety, e.g. an endoplasmic reticulum-targeting moiety as defined in any one of claims 7 to 10;
R³ is hydrogen, or an alkyl group such as C₁₋₃ alkyl (e.g. methyl);
each R⁴ is independently selected from C₁₋₆ alkyl, and —NR⁵R⁶;
R⁵ and R⁶ are independently selected from H and C₁₋₆ alkyl, preferably from H and C₁₋₃ alkyl (e.g. —CH₃); and
p is an integer from 0 to 3, preferably 0, 1 or 2, e.g. 0 or 1).

14. An agent for use as claimed in claim 13 which is a compound of formula (IIIa) or (IIIb): 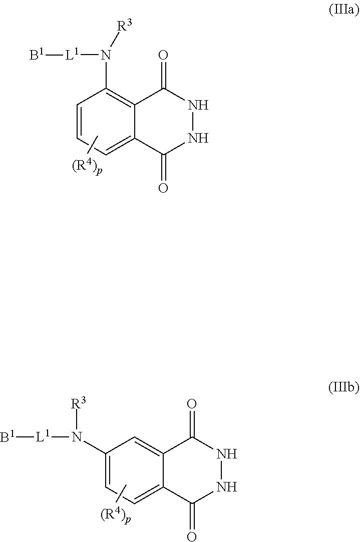
where L¹, B¹, R³, R⁴ and p are as defined in claim 13.

15. An agent for use as claimed in claim 13 or claim 14, wherein L¹ is selected from the group consisting of: 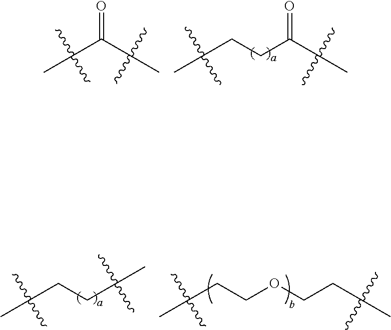
where a is an integer from 1 to 10, preferably from 3 to 10; and
b is an integer from 1 to 4, e.g. 2.

16. An agent for use as claimed in any one of claims 1 to 12 which is a compound of formula (IV), or a pharmaceutically acceptable salt thereof: 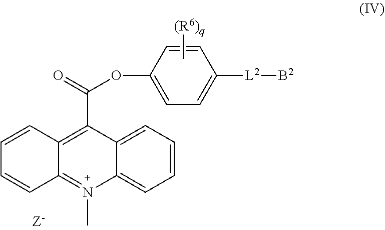
(where L² is a linker, e.g. a linker as defined in claim 11 or 12;
B² is an endoplasmic reticulum-targeting moiety, e.g. an endoplasmic reticulum-targeting moiety as defined in any one of claims 7 to 10;
each R⁶ is independently selected from halogen (e.g. F, Cl, Br, I), and C₁₋₆ alkyl (e.g. tert-Bu);
q is an integer from 0 to 4, preferably 0 or 2; and
Z is a monovalent anion, e.g. a Cl, Br, I, or CF₃OSO₂ anion).

17. An agent for use as claimed in claim 16, wherein L² represents one of the following groups: 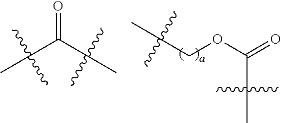
where a is an integer from 1 to 10, preferably 3, 4 or 5.

18. An agent for use as claimed in any one of claims 1 to 12 which is compound of formula (V), or a pharmaceutically acceptable salt thereof: 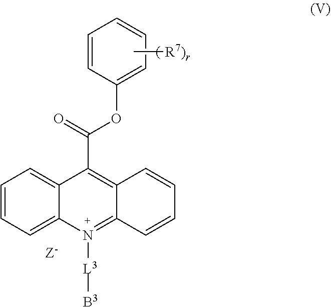
(where L³ is either a direct bond or a linker, e.g. a linker as defined in claim 11 or 12;
B³ is an endoplasmic reticulum-targeting moiety, e.g. an endoplasmic reticulum-targeting moiety as defined in any one of claims 7 to 10;
each R⁷ is independently selected from halogen (e.g. F, Cl, Br, I), —CO₂R⁸ (where R⁸ is hydrogen or C₁₋₆ alkyl), cyano, and C₁₋₆ alkyl (e.g. tert-Bu);
r is an integer from 0 to 5, preferably 0 or 3; and
Z is a monovalent anion, e.g. a Cl, Br, I, or CF₃OSO₂ anion).

19. An agent for use as claimed in claim 18, wherein L³ is a C₁₋₁₀ alkylene group, e.g. C₁₋₆ alkylene.

20. An agent for use as claimed in any one of claims 1 to 12 which is a compound of formula (VIa), (VIb), or a pharmaceutically acceptable salt thereof: 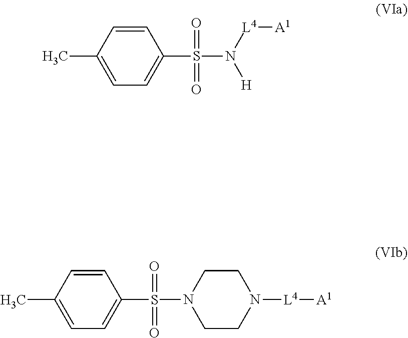
where L⁴ is either a direct bond or a linker, e.g. a linker as defined in claims 11 or 12; and
A¹ is a chemiluminescent moiety, e.g. a chemiluminescent moiety as defined in claim 5 or 6).

21. An agent for use as claimed in claim 20, wherein L⁴ is selected from the group consisting of: 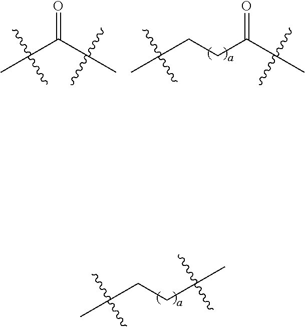
where a is an integer from 1 to 10, preferably from 3 to 10.

22. An agent for use as claimed in claim 20 or claim 21, wherein A¹ is selected from any of the following: 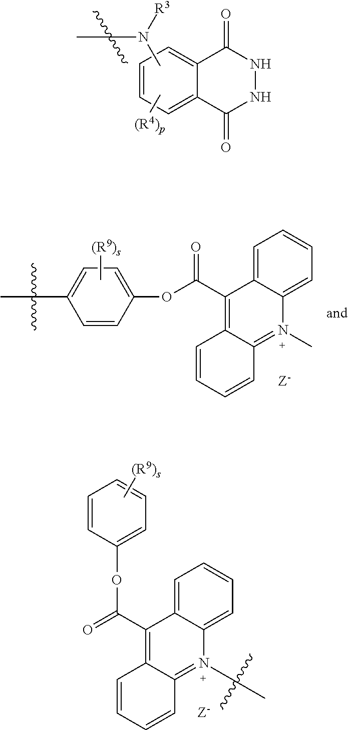
(where R³ is hydrogen, or an alkyl group such as C₁₋₃ alkyl (e.g. methyl);
each R⁴ is independently selected from C₁₋₆ alkyl, and —NR⁵R⁶;
R⁵ and R⁶ are independently selected from H and C₁₋₆ alkyl, preferably from H and C₁₋₃ alkyl (e.g. —CH₃);
p is an integer from 0 to 3, preferably 0, 1 or 2, e.g. 0 or 1;
Z is a monovalent anion, e.g. a Cl, Br, I, or CF₃OSO₂ anion;
each R⁹ is independently selected from halogen (e.g. F, Cl, Br, I) and C₁₋₆ alkyl (e.g. tBu); and
s is an integer from 0 to 4, preferably 0, 2 or 3).

23. An agent for use as claimed in any one of claims 1 to 12 which is a compound of formula (VII), or a pharmaceutically acceptable salt thereof: 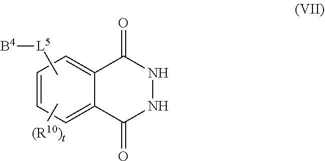
(where L⁵ is either a direct bond or a linker, e.g. a linker as defined in claim 11 or 12;
B⁴ is an endoplasmic reticulum-targeting moiety, e.g. an endoplasmic reticulum-targeting moiety as defined in any one of claims 7 to 10;
each R¹⁰ is independently selected from C₁₋₆ alkyl (e.g. methyl), and —NR¹¹R¹²;
R¹¹ and R¹² are independently selected from H and C₁₋₆ alkyl, preferably from H and C₁₋₃ alkyl (e.g. —CH₃); and
t is an integer from 0 to 3, preferably 1 or 2).

24. An agent for use as claimed in claim 23 which is a compound of formula (VIIa): 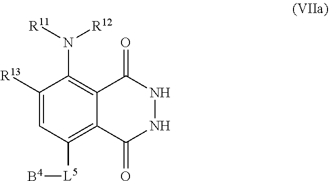
(where L⁵, B⁴, R¹¹ and R¹² are as defined in claims 23; and R¹³ is H or C₁₋₃ alkyl).

25. An agent for use as claimed in claim 23 or claim 24, wherein L⁵ is C₁₋₁₁ alkylene, preferably C₂₋₈ alkylene, e.g. propylene.

26. An agent for use as claimed in claim 1 selected from the following compounds:

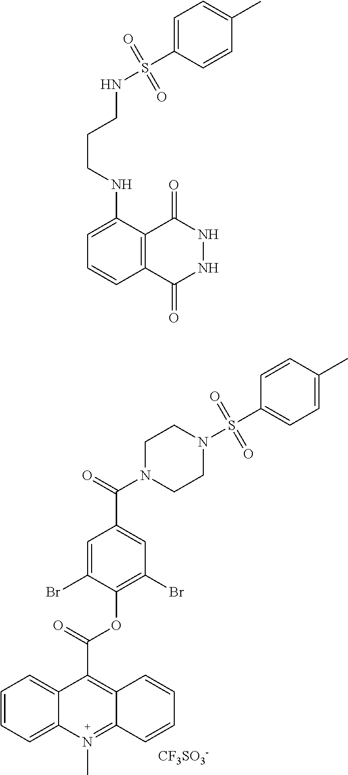

27. An agent for use as claimed in any one of the preceding claims, wherein said photodynamic therapy comprises simultaneous or sequential use of a photosensitizer or a precursor thereof.

28. An agent for use as claimed in claim 27, wherein said photosensitizer or precursor is selected from 5-aminolevulinic acid (5-ALA) and derivatives of 5-ALA, protoporphyrins (e.g. protoporphyrin IX); phthalocyanines such as metallated phthalocyanines which may optionally be sulphonated (i.e. AlPcS), e.g. di-sulphonated aluminium phthalocyanines such as AlPcS₂ or AlPcS₂ₐ, or aluminium phthalocyanine tetra-sulfonate (A1PcS₄); sulphonated tetraphenylporphyrins (e.g. TPPS₂ₐ, TPPS₄, TPPS₁ and TPPS₂ₒ); chlorins such as tetra(m-hydroxyphenyl)chlorins (m-THPC) (e.g. temoporfin which is marketed under the tradename Foscan); chlorin derivatives including bacteriochlorins and ketochlorins; mono-L-aspartyl chlorin e6 (NPe6) or chlorin e6; natural and synthetic porpyhrins including hematoporphyrin and benzoporphyrins; anthraquinones and derivatives thereof (e.g. hypericin, hypocrellins [A, B], cercosporin, calphostin, elsinochromes [A, B, C]).

29. An agent for use as claimed in claim 28, wherein said photosensitizer or precursor is selected from the following: cercosporin, 5-ALA, a derivative of 5-ALA, or a pharmaceutically acceptable salt thereof.

30. An agent for use as claimed in claim 28, wherein said photosensitizer is cercosporin.

31. An agent as claimed in any one of the preceding claims for use in the photodynamic treatment of any disorder or abnormality of cells or tissues in an animal body (e.g. a human) which is responsive to photodynamic therapy.

32. An agent for use as claimed in claim 31 in the treatment of cancer, preferably in the treatment of an internal cancer, e.g. a deeply-sited cancer.

33. An agent for use as claimed in claim 32, wherein said cancer is selected from the group consisting of gliomas and other brain cancers, hepatic and pancreatic cancers, breast, lung and prostate cancer, cholangiocarcinoma, stomach and colon cancers, bladder cancer, cervical cancers, head and neck cancers.

34. An agent for use as claimed in claim 33, wherein said cancer is GBM.

35. A pharmaceutical composition comprising an agent as defined in any one of claims 1 to 26, together with at least one pharmaceutically acceptable carrier or excipient.

36. A pharmaceutical composition comprising an agent as defined in any one of claims 1 to 26, and a photosensitizer or photosensitizer precursor as defined in any one of claims 27 to 30, together with at least one pharmaceutically acceptable carrier or excipient.

37. A pharmaceutical composition as claimed in claim 35 or claim 36 for use in photodynamic therapy, preferably for use in the treatment of an internal cancer, e.g. a deeply-sited cancer.

38. A product comprising an agent as defined in any one of claims 1 to 26, and a photosensitizer or photosensitizer precursor as defined in any one of claims 27 to 30 for simultaneous, separate or sequential use in a method of photodynamic therapy.

39. A kit comprising: (i) an agent as defined in any one of claims 1 to 26; and
separately (ii) a photosensitizer or photosensitizer precursor as defined in any one of claims 27 to 30; and optionally (iii) instructions for the use of (i) and (ii) in a method of photodynamic therapy.

40. Use of an agent as defined in any one of claims 1 to 26 in the manufacture of a medicament for use in combination therapy with a photosensitizer or photosensitizer precursor as defined in any one of claims 27 to 30, e.g. for use in a method of photodynamic therapy.

41. Use of a photosensitizer or photosensitizer precursor as defined in any one of claims 27 to 30 in the manufacture of a medicament for use in combination therapy with an agent as defined in any one of claims 1 to 26, e.g. for use in a method of photodynamic therapy.

42. Use of an agent as defined in any one of claims 1 to 26 together with a photosensitizer or photosensitizer precursor as defined in any one of claims 27 to 30 in the manufacture of a medicament for use in a method of photodynamic therapy.

43. A method of photodynamic therapy of cells or tissues of a patient (e.g. a human patient), said method comprising the step of administering to said cells or tissues:
(a) an effective amount of an agent as defined in any one of claims 1 to 26 and, simultaneously, separately, or sequentially thereto, an effective amount of a photosensitizer or photosensitizer precursor as defined in any one of claims 27 to 30; or (b) an effective amount of a pharmaceutical composition as defined in claim 36.

44. A conjugate as defined in any one of claims 1 to 26, or a pharmaceutically acceptable salt thereof.
